(19) United States
(12) Patent Application Publication
(10) Pub. No.: US 2021/0155561 A1
(43) Pub. Date: May 27, 2021

(54) METHOD AND REAGENT FOR DEOXYFLUORINATION

(71) Applicant: South Dakota Board of Regents, Pierre, SD (US)

(72) Inventors: Haoran Sun, Vermillion, SD (US); Siyu Mao, Vermillion, SD (US); Jordan H. Kramer, Vermillion, SD (US)

(73) Assignee: South Dakota Board of Regents, Pierre, SD (US)

(21) Appl. No.: 16/953,451

(22) Filed: Nov. 20, 2020

Related U.S. Application Data
(60) Provisional application No. 62/940,341, filed on Nov. 26, 2019.

(51) Int. Cl. C07B 39/00; C07D 495/04; C07D 495/14

(52) U.S. Cl. CPC C07B 39/00; C07D 495/14; C07D 495/04

(57) ABSTRACT

A safe, simple, and selective method and reagent for deoxyfluorination is disclosed. With the method and reagent disclosed herein, organic compounds such as carboxylic acids, carboxylates, carboxylic acid anhydrides, aldehydes, and alcohols can be fluorinated by using the most common nucleophilic fluorinating reagents and electron deficient fluoroarenes as mediators under mild conditions, giving corresponding fluoroorganic compounds in excellent yield with a wide range of functional group compatibility and easy product purification. For example, directly utilizing KF for deoxyfluorination of carboxylic acids provides the most economical and the safest pathway to access acyl fluorides, key intermediates for syntheses of peptide, amide, ester, and dry fluoride salts.

### RELATED APPLICATIONS

This application claims priority under 35 U.S.C. § 119(e) to U.S. Provisional Patent Application No. 62/940,341, filed Nov. 26, 2019, which is incorporated herein by reference.

### GOVERNMENT SUPPORT

This invention was made with government support under Grant No. CHE1355677 awarded by the National Science Foundation. The government has certain rights in the invention.

### BACKGROUND OF THE INVENTION

Owing unique physical, chemical, and biological properties, organofluorine compounds are vital for modern medicine and high performance materials. Developing new synthetic strategies and practices that utilize safe and commonly available, yet cost-effective, fluorinating reagents is the foundation for accessing a broader range of organofluorine compounds at different synthetic stages.

Also, fluorine is ubiquitous within medicinal and materials chemistries owing to the unique properties of organofluorine compounds. Substitution of compounds with fluorine or fluorinated moieties offers opportunities to tune the chemical and physical properties of the core structures. In medicinal chemistry, incorporation of fluorine and fluorinated moieties allows for increasing pharmaceutical durability due to the high stability of the C—F and corresponding C—C bonds. Fluorination also increases lipophilicity of drug molecules allowing for rapid uptake across membranes at targeted destinations therefore improving both the overall pharmacodynamics and pharmacokinetics.

While the incorporation of fluorine onto organic molecules can result in beneficial physicochemical property changes, significant challenges remain with the methodology and reagents available to accomplish fluorination in a safe and selective manner. Due to the wide availability of oxygen-containing functional groups, deoxyfluorination is a key fluorination strategy to prepare many fluoroorganics. DAST was first prepared in 1970 and was an alternative to the popular deoxyfluorinating reagent sulfur tetrafluoride. Despite deoxyfluorinating aliphatic alcohols, aldehydes, and unhindered ketones (Scheme 1A), as well as being a useful cyclization reagent; the thermal stability of DAST places serious restrictions on reaction conditions and safety protocols. Allowing DAST to heat up beyond 50° C. can result in the formation of the highly explosive diethylaminosulfur difluoride and highly toxic sulfur tetrafluoride. The danger posed by mishandling DAST prompted the research into developing several new deoxyfluorination reagents.

Over several decades, new deoxyfluorinating reagents have been prepared for use in research. Deoxo-Fluor® was an alternative deoxyfluorinating reagent, yet still displayed thermal instability resulting in accelerated decomposition above 60° C. XtalFluor-E® and XtalFluor-M® are two new deoxyfluorinating reagents in the form of crystalline solids and possess higher thermal stability, decomposing at 119° C. and 141° C., respectively. Both reagents possess higher stabilities and capabilities to deoxyfluorinate alcohols, aldehydes, and ketones (Scheme 1A), however these compounds impose a significant cost when needed on larger quantities. FluoLead™ possesses similar physical and chemical properties with a higher decomposition temperature of 170° C., yet suffers from both relatively high cost and less functional group tolerance.

Outside of industrial deoxyfluorinating reagents, two prime reagents were developed by the Ritter (Org. Lett. 2016, 18, 6102) and Sanford (Org. Lett. 2019, 21, 1350) groups. The Ritter group has developed a series of imidazolium-based reagents that are capable of performing phenol and heteroaromatic deoxyfluorination (Scheme 1B). These impressive results are further exemplified by the relative stability of the imidazolium salts in air and can be run at elevated temperatures. However, two disadvantages of PhenoFluor™ and its derivatives are the high reagent cost and availability of fluoride. The atomic economy is significantly hindered for large-scale production of fluorinated materials for pharmaceutical and materials manufacturing. Conversely, the Sanford group have developed an in situ deoxyfluorinating reagent capable of room temperature conversion of benzaldehydes and α-Ketoesters (Scheme 1C). The reagent sulfuryl fluoride was used to accomplish deoxyfluorination, yet is a toxic gas that still possesses safety concerns for both personnel and the environment.

Acyl fluorides are important carboxylic acid derivatives that are more stable and easier to handle than other acyl halides. Acyl fluorides are useful intermediates for the preparation of sterically and/or electronically challenging amides and esters, accessing aldehydes, ketones, and α,β-unsaturated carbonyl compounds, peptide syntheses, trifluoromethylarenes, and transition metal catalyzed coupling reactions. Acyl fluorides were recently used as starting materials to generate anhydrous fluoride salts in situ for S_{N}Ar fluorination reactions. Acyl fluorides can be prepared from acyl chlorides through chloride/fluoride exchange reactions with fluoride salts, preferably organic soluble anhydrous fluoride salts, for example, anhydrous tetrabutylammonium fluoride or tetramethylammonium fluoride. Directly preparing acyl fluorides from carboxylic acids and their salts is often highly attractive and chemically lucrative for syntheses of complex target compounds. Furthermore, methods for rapid late-stage fluorination of carboxylic acids with KF is expected to provide a new pathway to access [¹⁸F]-labeled acyl fluorides as radiotracers where a large selection of pharmaceuticals contain carboxylic acid functional groups and [¹⁸F]-KF is the most common [¹⁸F]-fluoride source produced in cyclotron target.

The first direct preparation of acyl fluorides from carboxylic acids was done by using cyanuric fluoride with Olah's seminal contribution in the early 1970s (Synthesis 1973, 487). Sulfur-based deoxyfluorinating reagents, for example, SF₄, DAST, Deoxo-Fluor®, XtalFluor-E®, XtalFluor-M®, and Fluolead™ were developed and used in deoxyfluorination of alcohols, aldehydes, ketones, and carboxylic acids and their derivatives over the past half century. However, serious safety and environmental precautions must be taken with when handling these reagents.

To overcome the explosive nature of sulfur-related deoxyfluorinating reagents such as DAST and Deoxo-Fluor®, a non-explosive pyridine-(HF)ₓ reagent with N,N′-dicyclohexylcarbodiimide was later used in deoxyfluorination and demonstrated limited functional group tolerance, though safety precautions on HF reagent usage along with environmental impacts of large scale usage of HF reagents must be taken into the consideration. While some of these recently developed deoxyfluorination reagents such as XtalFluor-E®, XtalFluor-M®, and Fluolead™ exhibit improved thermal stability, there are still rooms for improving the scope of the methodology, the cost-effectiveness of both reagent and the fluorine source. Schoenebeck et. al. recently broadened the utilization of an organic soluble tetramethylammonium trifluoromethanethiolate salt ([Me₄N]SCF₃), discovered by Tyrra et al. in 2003, to the preparation of acyl fluorides (J. Fluorine Chem. 2003, 119, 101). This procedure is, perhaps, more suitable for small quantity high value acyl fluoride preparation rather than large scale preparation as one of the side products is a toxic and flammable gas COS.

Recent work by Prakash utilizes an NBS/PPh₃/Et₃N-(HF)ₓ protocol, perhaps the most economical and safest reagents so far, for preparing acyl fluoride through deoxyfluorination of carboxylic acids (Org. Lett. 2019, 21, 1659). This latest deoxyfluorination protocol, however, only works under acidic condition where 2 eq. CF₃COOH is needed when using KF or KHF₂ as a fluorinating reagent. Lately, Paquin's group developed an alternative method to generate acyl fluorides by utilizing XtalFluor-E® and catalytic amount of NaF at room temperature with a typical 24 hrs reaction time to, perhaps, avoiding the high temperature decomposition of the XtalFluor-E® (J. Org. Chem. 2020, 85, 10253).

Accordingly, there is a need for a versatile fluorinating agent that is cost effective and compatible under a large variety of reaction conditions

### SUMMARY

This disclosure provides deoxyfluorination reagents and methods. The deoxyfluorination methods comprise the use of a fluoride salt and an electron-deficient fluoroarene. The deoxyfluoriantion reagent can be a composite reagent of a fluoride salt and an electron-deficient fluoroarene or heterofluoroarene. The scope of oxygen-containing substrates for the resulting fluorinated product is broad, numerous functional groups are tolerated, and the reaction has high selectivity.

In one embodiment, the invention provides a method for forming a fluorinated product comprising contacting an organic substrate, a fluoride salt, an electron-deficient fluorinated aromatic reagent, and a polar aprotic organic solvent, wherein the organic substrate comprises an oxygen moiety, typically a carbonyl or hydroxyl, to thereby form a C—F bond on the organic substrate.

In some embodiments, the method is carried out at above 20° C. In various embodiments, the organic substrate comprises an oxygen moiety that forms a C—O bond to the aromatic reagent via ipso substitution of an aromatic fluorine substituent of the aromatic reagent, and the C—O bond breaks via nucleophilic attack of fluoride ion to form a C—F bond, thereby forming the fluorinated product by deoxyfluorination of the organic substrate.

In various embodiments, the fluoroarene or fluoroaromatic is represented by Formula I or Formula IB:

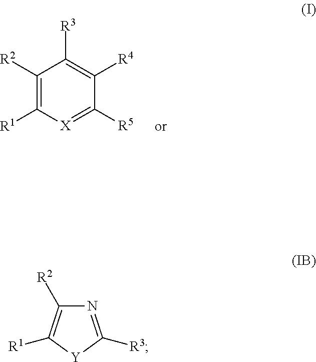

wherein

X is N or CR⁶ wherein R⁶ is F, CN, SF₅, or CₙF₂ₙ₊₁ wherein n is an integer equal to 1 or more;

Y is S or NR^{A} wherein R^{A} is H or —(C₁-C₆)alkyl;

R¹, R², R³, R⁴, and R⁵ are each independently F, CN, SF₅, or CₙF₂ₙ₊₁; or
R¹ to R⁶ are as defined above provided that R¹ and R² when taken together form a first heterocycle and R³ and R⁴ or R⁴ and R⁵ when taken together form a second heterocycle, wherein the first heterocycle and the second heterocycle are each independently an optional 5- or 6-membered heterocycle comprising an additional one or more EWG; and
   provided at least one of R¹ to R⁶ is fluoro.

Other various embodiments of this disclosure include a compound represented by Formula II or III:

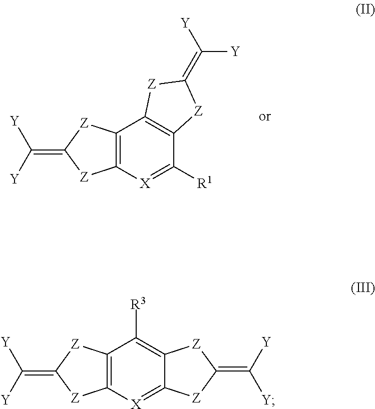

wherein

X is N or CR⁶;

R¹ or R³ is F, CN, SF₅, —OR⁷, or CₙF₂ₙ₊₁ wherein n is an integer equal to 1 or more;

R⁶ is F, CN, SF₅, —OR⁷, or CₙF₂ₙ₊₁;

R⁷ is saturated or unsaturated alkyl, acyl, aryl, or heteroaryl;

each Y is independently fluoro or cyano;

each Z is independently O or S; and

provided at least one of R¹ or R⁶ is F or OR⁷, or one of R³ or R⁶ is F or OR⁷.

### DETAILED DESCRIPTION

A safe, simple, and selective method and reagent for deoxyfluorination is disclosed. With the method and reagent disclosed herein, organic compounds such as carboxylic acids, carboxylates, carboxylic acid anhydrides, aldehydes, alcohols, phenols, epoxides etc. can be fluorinated by using a composite fluorinating reagent that comprises the most common fluoride salts, including but not limited to KF, CsF, NH₄F, tetramethylammonium fluoride (TMAF), tetraethylammonium fluoride (TEAF), tetrapropoalammonium fluoride (TPAF), tetrabutylammonium fluoride (TBAF), tetrahexylammonium fluoride (THAF), N,N,N-trialkylanilinium fluorides, cobaltocenium fluoride (CoCp₂F), and tetrabutylammonium difluorotriphenyl silicate (TBAT), and electron deficient fluoroarenes with electron-deficiency greater than fluorobenzonitrile as mediators under mild conditions, giving corresponding deoxyfluorinating products such as acyl fluorides, difluoromethylated, and monofluorinated, 1,2-difluoroethyl fluoroorganic compounds in excellent yield with a wide range of functional group compatibility and easy product purification. For example, directly utilizing KF for deoxyfluorination of carboxylic acids provides the most economical and the safest pathway to access acyl fluorides, key intermediates for syntheses of peptide, amide, ester, and dry fluoride salts. Under optimal condition, this deoxyfluorination strategy can be a method of choice for rapid late stage fluorination of pharmaceutically important compounds which is significant for preparing [¹⁸F] labeled radiotracers.

Herein, we report a new method for the synthesis of acyl fluorides from carboxylic acids using KF and highly electron-deficient fluoroarenes. Notably, this safe method shows not only high temperature tolerance up to 180° C., but broad reaction scope including pharmaceuticals. Furthermore, fast reactions (within 8 mins) and large-scale synthesis shows this methodology is ideal for both academic and industrial applications.

Also, we report a new selective deoxyfluorination procedure that is not only safe for its user and environment, but also widely available from commercial materials, making the methodology applicable for laboratory- and industrial-scale production of fluorinated materials.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture.

An “effective amount” refers to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

As used herein, the term “substituted” or “substituent” is intended to indicate that one or more (for example., 1-20 in various embodiments, 1-10 in other embodiments, 1, 2, 3, 4, or 5; in some embodiments 1, 2, or 3; and in other embodiments 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable substituents, variable groups, or indicated groups include, e.g., alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, or cyano. In various embodiments, a substituent can be any one or more of the aforementioned groups.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like.

The term “heterocycloalkyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aromatic” refers to either an aryl or heteroaryl group (e.g., fluroroheteroaromatic) or substituent described herein. Additionally, an aromatic moiety may be a bisaromatic moiety, a trisaromatic moiety, and so on. A bisaromatic moiety has a single bond between two aromatic moieties such as, but not limited to, biphenyl, or bipyridine. Similarly, a trisaromatic moiety has a single bond between each aromatic moiety.

The term “aryl” or “arene” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms.

The term “heteroaryl” refers to a monocyclic, bicyclic, or tricyclic ring system containing one, two, or three aromatic rings and containing at least one nitrogen, oxygen, or sulfur atom in an aromatic ring. Examples include the heteroaryl ring skeletons and structures of Formula I (when X is N), Formula II, and Formula III, as well as heteroaryl compounds 1-4 described in Example 1.

Alkyl, cycloalkyl, heterocycloalkyl, aryl, and heteroaryl groups can be unsubstituted, or substituted with one or more substituents (e.g., 1-6, 1-5, 1-4, 1-3, or 1 or 2), for example, one or more substituents described in the definition of “substituted”.

### Embodiments of the Invention

This disclosure provides a compound represented by Formula I or IB:

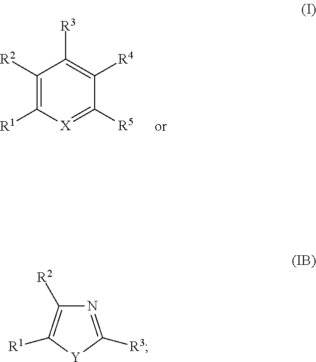

wherein

X is N or CR⁶ wherein R⁶ is F, CN, SF₅, or CₙF₂ₙ₊₁ wherein n is an integer equal to 1 or more;

Y is S or NR^{A} wherein R^{A} is H or —(C₁-C₆)alkyl;

R¹, R², R³, R⁴, and R⁵ are each independently F, CN, SF₅, or CₙF₂ₙ₊₁; or
R¹ to R⁶ are as defined above provided that R¹ and R² when taken together form a first heterocycle and R³ and R⁴ or R⁴ and R⁵ when taken together form a second heterocycle, wherein the first heterocycle and the second heterocycle are each independently an optional 5- or 6-membered heterocycle comprising an additional one or more EWG; and provided at least one of R¹ to R⁶ is fluoro.

Also, this disclosure provide compound represented by Formula II or III:

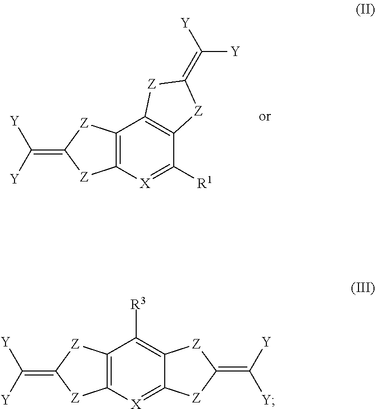

wherein

X is N or CR⁶;

R¹ or R³ is F, CN, SF₅, —OR⁷, or CₙF₂ₙ₊₁ wherein n is an integer equal to 1 or more;

R⁶ is F, CN, SF₅, —OR⁷, or CₙF₂ₙ₊₁;

R⁷ is saturated or unsaturated alkyl, acyl, aryl, or heteroaryl;

each Y is independently fluoro or cyano;

each Z is independently O or S; and

provided at least one of R¹ or R⁶ is F or OR⁷, or one of R³ or R⁶ is F or OR⁷.

In various embodiments, the compound is an electron-deficient fluoroaromatic. In some embodiments, the compound is a fluorophenylnitrile or a fluoroarene. In other embodiments, the compound is a tetrafluorophthalonitrile. In other embodiments, the compound is 3,4,5,6-tetrafluorophthalonitrile (TFPN). In some embodiments, the scope of the compounds disclosed herein excludes prior known compounds.

In various embodiments, the compound of Formula I is represented by Formula II:

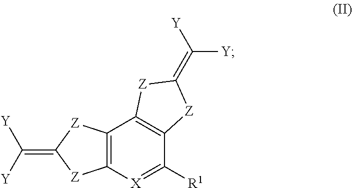

wherein

each Y is independently fluoro or cyano; each Z is independently O or S; and
at least one of R¹ or R⁶ is F.

In various embodiments, the compound is 2,2′-(4-cyano-5-fluorobenzo[1,2-d:3,4-d′]bis([1,3]dithiole)-2,7-diylidene)dimalononitrile:

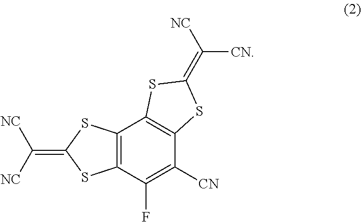 [SMILES: N#CC(C#N)=C1Sc2c(F)c(C#N)c3c(c2S1)SC(=C(C#N)C#N)S3]

In various embodiments, the compound is 2,2′-(5-fluorobis([1,3]dithiolo)[4,5-b:4′,5′-d]pyridine-2,7-diylidene)dimalononitrile:

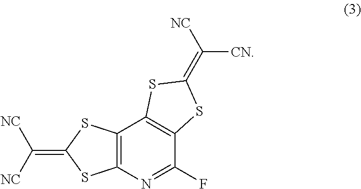 [SMILES: N#CC(C#N)=C1Sc2nc(F)c3c(c2S1)SC(=C(C#N)C#N)S3]

In various embodiments, the compound of Formula I is represented by Formula III:

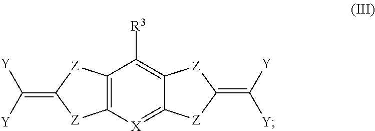

wherein

each Y is independently fluoro or cyano;

each Z is independently O or S; and
at least one of R³ or R⁶ is F.

In various embodiments, the compound is 2,2′-(4-cyano-8-fluorobenzo[1,2-d:4,5-d′]bis([1,3]dithiole)-2,6-diylidene)dimalononitrile:

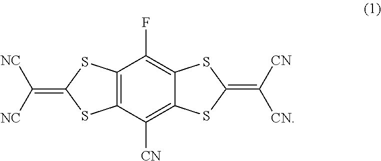 [SMILES: N#CC(C#N)=C1Sc2c(F)c3c(c(C#N)c2S1)SC(=C(C#N)C#N)S3]

In various embodiments, the compound is 2,2′-(8-fluorobis([1,3]dithiolo)[4,5-b:4′,5′-e]pyridine-2,6-diylidene)dimalononitrile:

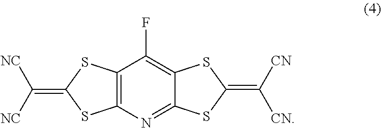 [SMILES: N#CC(C#N)=C1Sc2nc3c(c(F)c2S1)SC(=C(C#N)C#N)S3]

Additionally, this disclosure provide a composition of the compound of Formula I, II, or III and a fluoride salt. In other embodiments, this disclosure provides a composition comprising a fluoride salt and an electron-deficient fluoroarene. In some embodiments, this disclosure provides use of fluoride salt and an electron-deficient fluoroarene.

Furthermore, this disclosure provides a method for deoxyfluorination comprising contacting an organic substrate, a fluoride salt, an electron-deficient fluoroaromatic, and an organic solvent at a reaction temperature above −80° C.;

wherein the electron-deficient fluoroaromatic comprises one or more additional electron withdrawing group (EWG);

the substrate and fluoroaromatic form an intermediate;

the substrate comprises an oxygen moiety, the oxygen moiety forms an intermediate C—O bond via ipso substitution of a fluoro substituent on the aromatic moiety of the fluoroaromatic; and

the intermediate C—O bond breaks via nucleophilic attack by a fluoride ion at the substrate moiety of the intermediate to form a C—F bond;

wherein a fluorinated product is thereby formed by deoxyfluorination of the organic substrate.

In various embodiments, the electron-deficient fluoroaromatic is an electron-deficient fluoroheteroaromatic that has at least one fluorine atom and one or more (additional) electron withdrawing groups including F, CN, CF₃, SF₅, S₂C(CN)₂, S₂C═C(CN)₂, or CₙF₂ₙ₊₁, wherein n is an integer equal to or greater than 1.

In various embodiments, integer “n” of the electron withdrawing group CₙF₂ₙ₊₁ is equal to or greater than 1. For example, n can be 1-20, 1-12, 1-10, 1-8, 1-6, 1-4, 1-3, 2-20, 2-12, 2-10, 2-8, 1-6, 2-4, or 2-3. In some embodiments, integer n of CₙF₂ₙ₊₁ is 1, 2, 3, 4, 5, 6, 7, 8, 9, 10, 11, or 12, or any combination thereof. In one specific embodiment, integer n is 1 (i.e., the group is —CF₃). In another specific embodiment, integer “n” is 2 (i.e., the group is —CF₂CF₃).

In some embodiments, the organic solvent is polar. In some embodiments, the organic solvent is aprotic. In other embodiments the organic solvent is polar and aprotic. In other embodiments, the organic substrate comprises an alcohol, phenol, aldehyde, carboxylic acid, epoxide, or salt thereof. In other embodiments, the fluoride salt is lithium fluoride, sodium fluoride, potassium fluoride, cesium fluoride, ammonium fluoride, tetramethylammonium fluoride, tetraethylammonium fluoride, tetrapropylammonium fluoride, tetrabutylammonium fluoride, or a fluoride isotope thereof (e.g., for preparing [¹⁸F] labeled radiotracers).

In some embodiments, the organic solvent is acetonitrile, N-methyl-2-pyrrolidinone, propylene carbonate, dimethyl sulfoxide, tetrahydrofuran, or benzonitrile. In some embodiments, the reaction temperature is about −20° C. to about 190° C., about 0° C. to about 190° C., about 0° C. to about 120° C., about 25° C. to about 100° C., about 50° C. to about 100° C., or about 100° C. to about 190° C. In some embodiments, the reaction temperature is 200° C. or lower. In some embodiments, the product formed comprises an alkyl fluoride, acyl fluoride, aryl fluoride, or heteroaryl fluoride. In some embodiments, the product formed comprises a difluoromethyl group.

In some embodiments, the fluorinated aromatic reagent is 3,4,5,6-tetrafluorophthalonitrile, the fluoride salt is potassium fluoride or tetramethylammonium fluoride, the organic solvent is acetonitrile, and the reaction temperature is about 65° C. to about 105° C., or about 75° C. to about 95° C.

Deoxyfluorination of Carboxylic Acids with KF and Highly Electron-Deficient Fluoroarenes

### Results and Discussion

A common key step for carboxylic acid deoxyfluorination is the removal of OH⁻, by forming a strong X═O bond (X=S, P, C) in the side product during the reaction. We hypothesize that removal of OH⁻ can also be done if we convert this abysmal leaving group into a relatively better leaving group where the negative charge on oxygen is stabilized by electron-withdrawing moieties and delocalization through resonance. A key strategy in our deoxyfluorination approach is to prepare an electron-deficient phenoxide as a better leaving group by using fluorinated electron-deficient aromatics as reaction mediators (Scheme X). The fluorine bearing ipso-carbon in the highly electron-deficient fluoroarene is susceptible to nucleophilic attack even with relatively weak nucleophiles, here, the carboxylates, forming esters (Scheme X, 1^{st} step). Then the highly reactive nucleophile, fluoride (anhydrous form), attacks the carbonyl carbon to form a C—F bond and break the ester C—O bond, forming electron deficient phenoxide (Scheme X, 2^{nd} step) as a better leaving group.

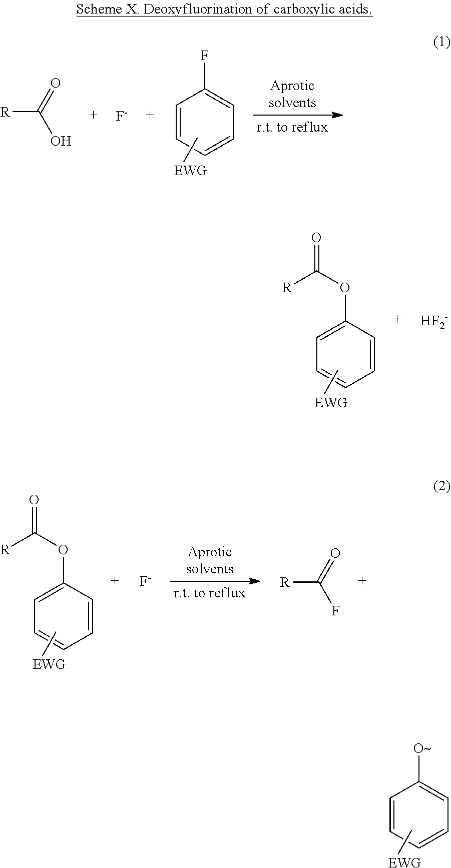

To test our hypothesis, we chose a simple highly electron-deficient fluoroaromatic, tetrafluorophthalonitrile (TFPN), as a reaction mediator that facilitates the formation of an ester intermediate in the first step and the formation of a better leaving group in the second step in Scheme X. Two important factors we took into consideration for using TFPN are: 1) the phenyl ring is highly electron-deficient due to the strong electron withdrawing effects of both multiple fluorines and two cyano groups, leading to a faster S_{N}Ar reaction in the first step and forming a better leaving group in the second step; 2) there is no acidic proton that can consume the highly reactive fluoride. Together with spray-dried KF, direct deoxyfluorination of carboxylic acids was achieved.

In a typical reaction, a carboxylic acid was mixed with 2.5 eq. spray-dried KF and 1.2 eq. TFPN in anhydrous acetonitrile and the resulting mixture was heated at 80° C. The reaction was monitored by both ¹H and ¹⁹F NMR spectra periodically and final reaction yield was determined by integration of ¹H and/or ¹⁹F NMR spectra by adding TBAPF₆ as an internal standard as well as isolate yield for some pharmaceutical products. Upon heating, the reaction typically finished within overnight. Varying the amount of TFPN being used in the reaction, we found that 1 equiv. of TFPN is needed. Given that TFPN also reacts with water to form corresponding phenoxide, a competing reaction for the S_{N}Ar reaction in the first step if the sample contains trace amounts of residual water, we utilize a slight excess TFPN (1.2 equiv.) in practice except noted otherwise.

TABLE 1
Examples of reaction mediators.

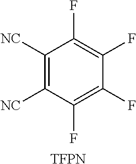

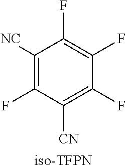

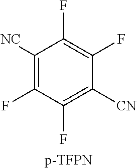

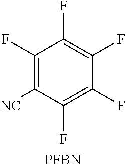

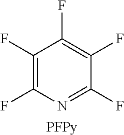

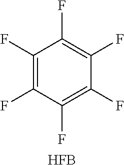

Several other commercially available electron-deficient fluoroarenes that do not possess active proton were also examined as reaction mediators (Table 1). Using para-N,N-dimethylamino-benzoic acid as an example, we tested six different electron-deficient fluoroarenes under the same condition. Except HFB, all of these electron-deficient fluoroarenes work for the deoxyfluorination reaction, giving similar result compared to TFPN. Using a relatively milder electron-deficient fluoroarene than HFB (e.g. para-fluorobenzonitrile, as the reaction mediator), the above reaction did not occur. Though searching for an electron-deficiency threshold for other fluoroarene mediators is theoretically important, we feel that reporting this safe, simple, and cost-effective practical method first will benefit broader synthetic and medicinal chemistry communities. More reactive anhydrous fluoride salts (e.g. anhydrous tetramethylammonium fluoride (TMAF)) also can be used as the fluoride source to generate the target acyl fluoride product. However, the cost is much higher than that of spray-dried KF. Considering the reagent cost and easy to handle practice, we chose TFPN as the reaction mediator and spray-dried KF as the fluorinating reagent for the rest of the study in this report. The TFPN/KF combination holds a significant benefit, thermal stability, over those highly reactive deoxyfluoriantion reagents, like SF₄, DAST, Deoxo-Fluor®, XtalFluor-E®, XtalFluor-M®, and Fluolead™. In addition, the deoxyfluorination reaction with TFPN/KF reagent does not generate any decomposition gas product like TMA[SCF₃] does, resulting much safer operation even at large scale.

The presented deoxyfluorination strategy and reagents worked for both aliphatic and aromatic carboxylic acids with various functional groups (Table 2). The reaction condition is tolerant to many functional groups, including cyano, trifluoromethyl, aryl halides, ketone, aldehyde, ether, ester, alkyne, alkene, and amine. A key advantage of this TFPN/KF method for acyl fluoride preparation is shown by the high tolerance of the aldehyde and ketone functional groups. This is unlike other traditional deoxyfluorination reagents like SF₄, DAST, XtalFluor® react with aldehydes and ketones as well. Such selectivity is particularly important for late-stage fluorination when multiple functional groups are coexisted. Generally, the reaction yield ranges from 34% to 92% without further optimization of experimental conditions (Table 2 and Table 3). Aliphatic carboxylic acids including sterically hindered carboxylic acids gave good yields. Substituted benzoic acids, with both electron-rich and electron-poor phenyl rings, give good to excellent yields (60-92%). It seems the electronic effects of the substituents on the phenyl rings do not have an observable impact on the product yields under current reaction conditions, though those substituents may affect the reaction kinetics. Substrates having amine groups made a slightly complicated case as the amine group could react with the acyl fluoride to form amide-based polymers.

Monitoring the reaction by ¹H and ¹⁹F NMR followed by quenching at the appropriate time gives us about 62% yield of para-aminobenzoyl fluoride. Due to its nucleophilicity, the hydroxyl group on benzoic acid becomes a competitor in the first esterification step, resulting in a complex mixture when 1.2 equiv. TFPN is used. When excess TFPN (2.2 equiv.) and KF (3.5 equiv.) were used, the reaction with hydroxyl substituted benzoic acid went to completion with the hydroxyl group being converted into trifluoro-dicyano-phenoxyl group on the corresponding acyl fluoride product in 72% yield. Both nicotinic and isonicotinic acids were also converted to the corresponding acyl fluorides in good yield, 77% and 68%, respectively.

To illustrate the synthetic scope of this method, we further demonstrated this deoxyfluorination strategy with several pharmaceutical compounds that contain carboxylic acid groups, giving moderate isolated yield (34-47%) of corresponding acyl fluorides (Table 3). For this group of substrates typically we used 0.5 mmol to 4.0 mmol scale of substrates, 1.05 equiv. TFPN and 2.5 equiv. KF with appropriate solvents depending reaction temperature (see experimental section for details). While, in CH₃CN solution at 80° C., the deoxyfluorination reactions for these pharmaceutical compounds are still too slow for using in [¹⁸F]-fluoride labeling reactions due to its short lifetime, a half-life of 109.5 minutes. To shorten the reaction time, a higher boiling point aprotic solvent, propylene carbonate (PC), was employed for rapid fluorination of these pharmaceutical compounds at 180° C. Under this condition a benchmark example, para-dimethylamino-benzoic acid, was fluorinated with 70% yield in about 15 minutes. In a PC solution at 180° C., a nonsteroidal anti-inflammatory drug Naproxen, an inhibitor for cyclooxygenase-1 and -2, was fluorinated within 8 minutes with 42% isolated yield. Other pharmaceutical compounds containing sulfonamide, ketone, nitrile functional groups and heterocycles were fluorinated under the same condition giving 47-76% yield.

These successful late-stage fluorination results demonstrated a potential possibility of labeling those drug molecules with [¹⁸F]-KF. Both pharmaceutical compounds (Naproxen and Febuxostat) demonstrated high compatibility with the high temperature reaction condition with 40% isolated yield. Furthermore, the scalability of this method has been demonstrated by directly using a pharmaceutical compound, Probenecid, with 4 mmol scale (1.14 g) and obtained in 34% isolated yield. The relatively low isolation yields we observed are, perhaps, due to the instability of the acyl fluoride under the isolation condition; and potential loss during the crystallization process. Searching for more reactive mediators that could further increase the reaction rate at lower temperature is current underway in this laboratory.

TABLE 2
List of deoxyfluorination products and yields.

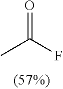

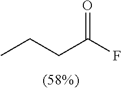

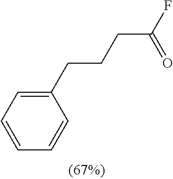 [SMILES: O=C(F)CCCc1ccccc1]

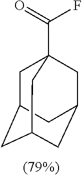 [SMILES: O=C(F)C12CC3CC(CC(C3)C1)C2]

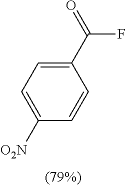 [SMILES: O=C(F)c1ccc([N+](=O)[O-])cc1]

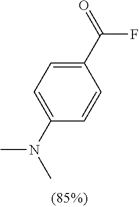

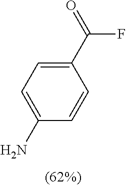

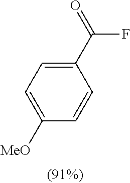 [SMILES: COc1ccc(C(=O)F)cc1]

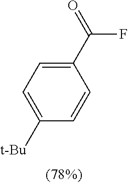 [SMILES: CC(C)(C)c1ccc(C(=O)F)cc1]

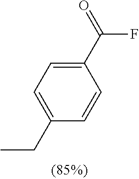

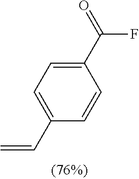

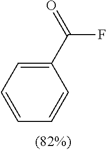

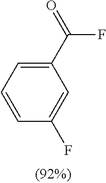

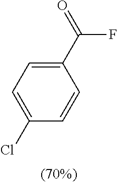

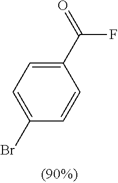

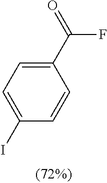

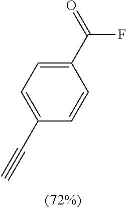

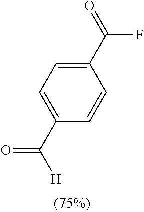 [SMILES: O=Cc1ccc(C(=O)F)cc1]

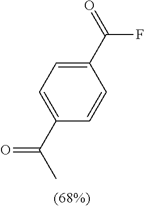

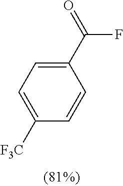 [SMILES: O=C(F)c1ccc(C(F)(F)F)cc1]

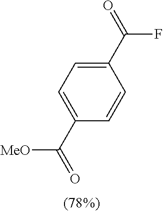 [SMILES: COC(=O)c1ccc(C(=O)F)cc1]

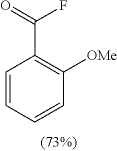 [SMILES: COc1ccccc1C(=O)F]

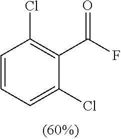 [SMILES: O=C(F)c1c(Cl)cccc1Cl]

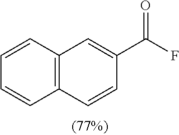 [SMILES: O=C(F)c1ccc2ccccc2c1]

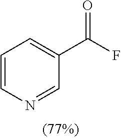

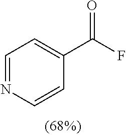

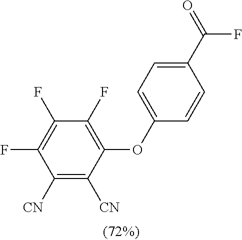

Yield was determined by ¹⁹F NMR with the internal standard TBAPF₆.

TABLE 3
Fluorinated pharmaceutical compounds.

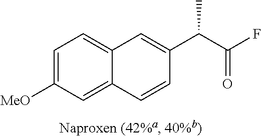 [SMILES: COc1ccc2cc([C@H](C)C(=O)F)ccc2c1]

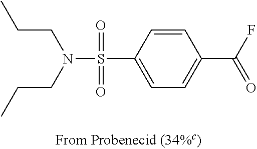 [SMILES: CCCN(CCC)S(=O)(=O)c1ccc(C(=O)F)cc1]

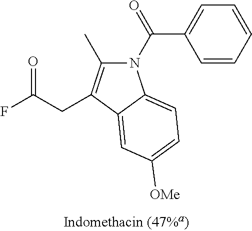 [SMILES: COc1ccc2c(c1)c(CC(=O)F)c(C)n2C(=O)c1ccccc1]

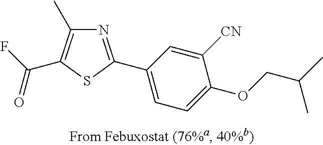 [SMILES: Cc1nc(-c2ccc(OCC(C)C)c(C#N)c2)sc1C(=O)F]

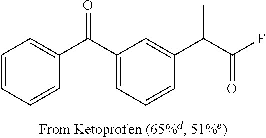 [SMILES: CC(C(=O)F)c1cccc(C(=O)c2ccccc2)c1]

^{a}Isolated yield with reaction condition of heating at 80° C. in CH₃CN for 24 hrs;
^{b}Isolated yield with reaction condition of heating at 180° C. in PC for 8 mins;
^{c}1 g scale isolated yield with reaction condition of heating at 80° C. in CH₃CN for 24 hrs;
^{d}NMR quantified yield under the condition of heating at 80° C. in CH₃CN for 24 hrs;
^{e}NMR quantified yield under the condition of heating at 180° C. in PC for 8 mins.

Converting an “impossible” leaving group into a better leaving group is the key for this successful deoxyfluorination strategy. The leaving-ability of the corresponding phenoxide is mainly determined by its stability, or the acidity of its conjugate acid. Our initial DFT calculation of the gas phase proton affinity of the corresponding phenoxide from TFPN is about −293 kcal/mol compared to non-substituted phenoxide of −344 kcal/mol at room temperature. We estimated that the pKa of corresponding dicyano-trifluoro-phenol is about 37 pKa unit lower than that of non-substituted phenol in gas phase. With IEFPCM solvation model, it is about 16 and 15 pka unit lower than that of phenol in acetonitrile and water, respectively. These results indicate that dicyano-trifluoro-phenol is a very strong acid, resulting in the corresponding phenoxide as an excellent leaving group. Here, three fluorine and two cyano substituents serve the function of electron withdrawing and charge delocalization through resonance. There are still demands to further investigate and improve the mediator's S_{N}Ar reactivity (step 1 in Scheme X) and corresponding phenoxide leaving group-ability by introducing even stronger electron-withdrawing groups with better resonance onto the mediators. Practically speaking, TFPN is so far the most economical reaction mediator commercially available and works to generate a superior leaving group for this deoxyfluorination reaction.

In summary, we have successfully demonstrated a safe, cost-effective, and chemoselective deoxyfluorination strategy for preparing acyl fluorides from carboxylic acids in good to excellent yields. Excellent functional group compatibility opens a door for expanding the method into direct fluorination of many substrates including current drugs or drug candidates containing carboxylic acid groups. Rapid fluorination of several pharmaceutical compounds in good yield within 8 mins promises a great potential for new [¹⁸F]-fluorinated radiotracer synthesis while searching for new mediators that can eliminate the [¹⁸F] and [¹⁹F] isotope exchange is on the way.

### Deoxyfluorination of Aldehydes, Alcohols, and Phenols Utilizing an Electron-Deficient Fluoroaromatic and Tetramethylammonium Fluoride

### Results and Discussion

Our approach on developing a new deoxyfluorination method for aldehydes, alcohols, and phenols is based on converting atrocious leaving groups into superior leaving groups, similar to the DAST deoxyfluorination mechanism. Our experiments focused on finding a commercially available reagent that would be low in cost but capable of the leaving group conversion. Tetrafluorophthalonitrile (TFPN) and the tetrafluoroisophthalonitrile (iso-TFPN) were chosen due to their electron-deficient aromatic rings providing multiple S_{N}Ar sites. Both TFPN and iso-TFPN additives were successful in converting aromatic aldehydes, benzyl alcohols, and some phenols into their deoxyfluorinated counterparts (Scheme 1D).

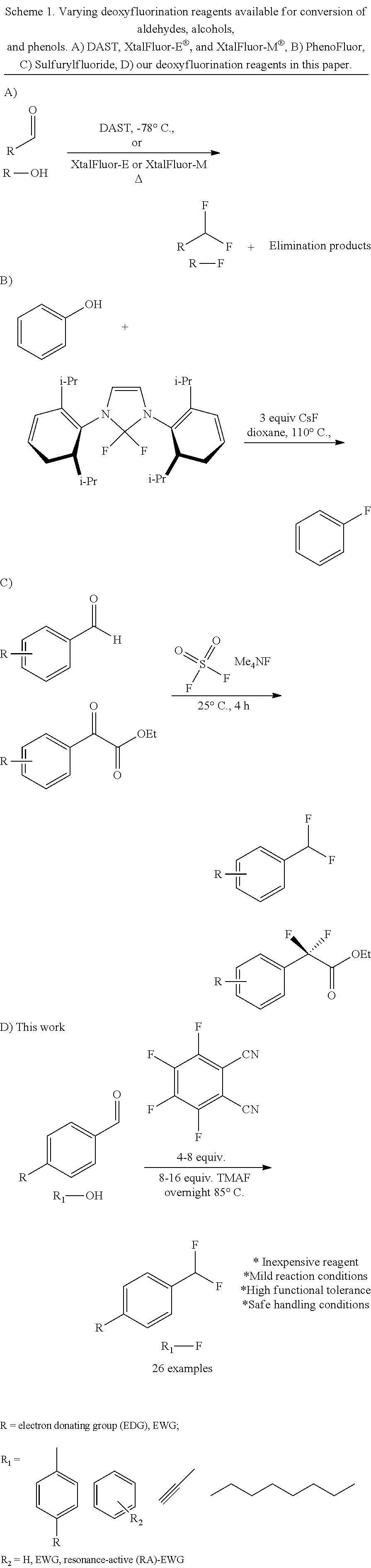

N,N-dimethylaminobenzaldehyde (0.062 mmol) was added into a small vial containing TFPN (0.25 mmol) and anhydrous TMAF (0.5 mmol) and 0.5 ml of CH₃CN (dried over flame-dried 4 Å molecular sieves and alumina) under inert atmosphere. The resulting reaction mixture was sealed in an NMR tube, transferred out of the glovebox and heated to 85° C. for overnight. The reaction mixture was cooled down to room temperature; no-D NMR experiments were obtained to assess the formation of the products. Then, methylene chloride was added and the resulting mixture was filtered. The reaction yield was determined as 85% by GC-MS measurement of the corresponding filtrate. Both substrates with electron donating groups (EDG) and electron withdrawing groups (EWG) provided good to excellent yields with overnight heating and excess TFPN and TMAF (Scheme 2); the lone exception was the methoxy substitution, giving a fair 45% yield without further reaction condition optimization. Many active functional groups such as nitrile, dimethyl amino, ether, ester, and alkenyl are tolerated under the reaction conditions. The hydroxyl group was converted into the corresponding ether through a S_{N}Ar reaction with TFPN. The conversion of heterocyclic aldehydes was met with mixed results. 3-Formylthiophene was able to achieve a good yield to the germinal difluoride, while the 2-formylpyridine demonstrated a quantitative yield.

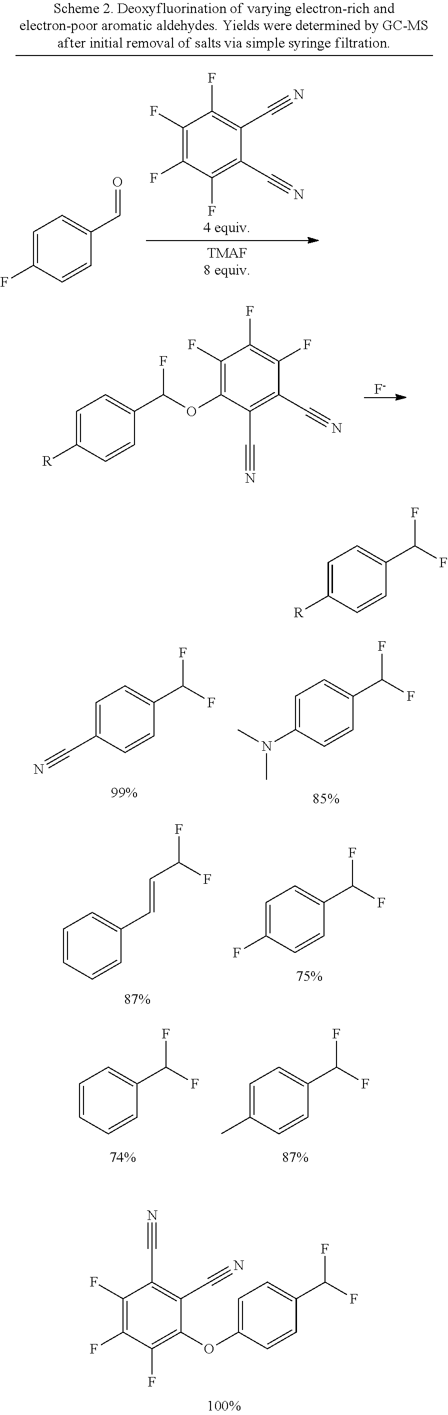

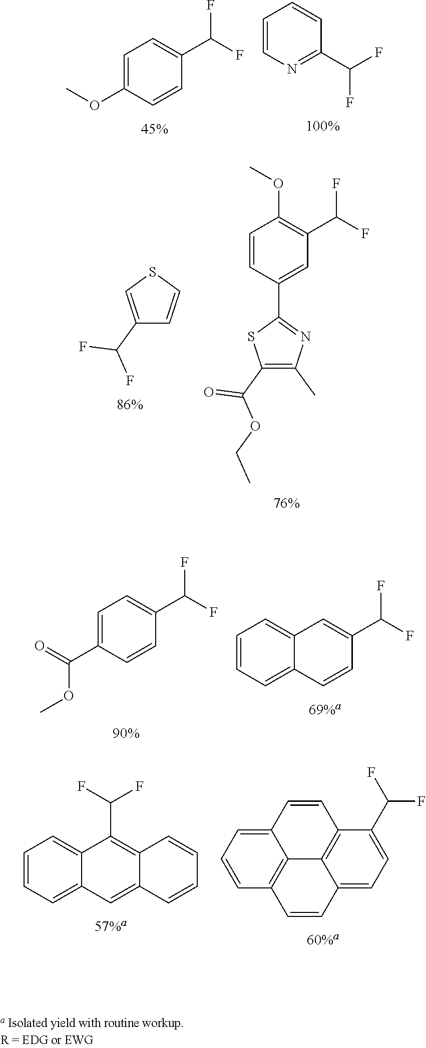

A major challenge within the aldehyde deoxyfluorination reactions was the presence of residual water in both substrates and reagents. The yields reported in Scheme 2 are from substrates soaked in flame-dried alumina for 2 days. The residual water quickly consumed fluoride to generate bifluoride, which was observed to grow over time due to a broadening signal near −160 ppm indicating a possible anion-π interaction taking place between TFPN and bifluoride. In a control experiment, water was purposely added into the mixture of TFPN and anhydrous TMAF solution without substrate. The active anhydrous fluoride was then consumed by water to form bifluoride before interacting with TFPN to produce a similar broad peak around −160 ppm. In contrast, anhydrous TMAF and TFPN mixture showed a broad signal in the range of −120-135 ppm due to the fluoride-π interaction. These results indicate that one can either further dry the substrates or add excess TFPN and anhydrous TMAF to remove residual water in the reaction mixture to further improving the reaction yield.

In addition to the deoxyfluorination of aldehydes, a selection of alcohols were investigated to observe the potential deoxyfluorination capability of the TFPN/TMAF reagent. These alcohols consist of a primary alcohol, benzylic alcohols substituted with electron donating and electron withdrawing groups, and propargyl alcohol (Scheme 3). The yields for the alcohol conversion were impressive; all benzyl alcohols were fully converted to their corresponding fluoromethyl derivatives based on GC-MS. This high conversion was also carried over to the propargyl alcohol trial, through the sample was only characterized via NMR due to the volatile nature of the subsequent fluorinated product. Interestingly, 1-octanol showed a preference for undergoing deoxyfluorination over the typical elimination pathway. Even with heating at 85° C. overnight, the yield of 1-fluorooctane was 92% and 8% 1-octanene based on GC-MS. Secondary alcohol trials mainly lead to the elimination products, alkenes, due to the strong basicity of the fluoride itself in anhydrous aprotic solvents. Restricting the basicity of the reagents is likely a key factor for selective deoxyfluorination of secondary alcohols while preventing elimination. Regardless of the deoxyfluorination results with secondary alcohols, these promising results show the potential of TFPN and similar electronically deficient aromatics in deoxyfluorination in comparison to the limitations imposed by DAST.

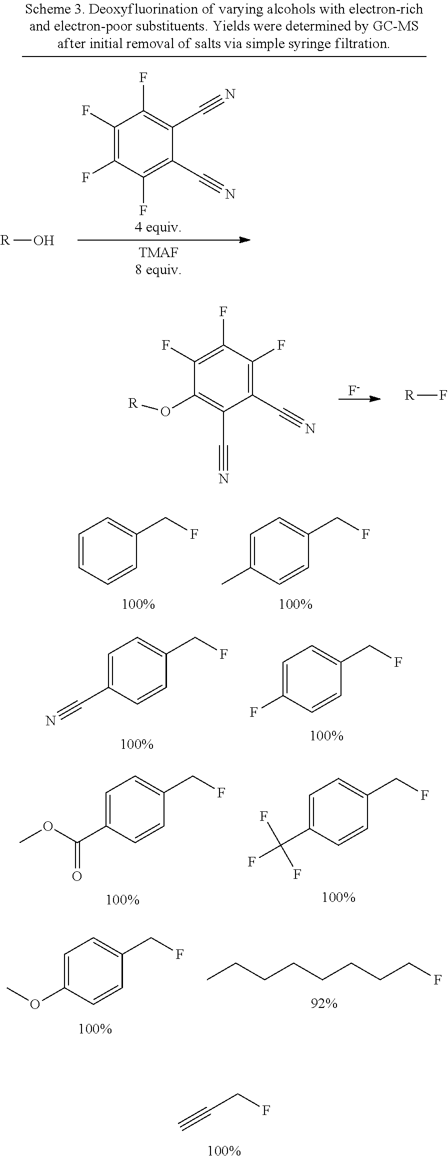

The success of benzylic alcohols and octanol prompted us to explore one of the significant hurdles in deoxyfluorinations: phenol deoxyfluorination. Perhaps one of the most surprising results came from our trial reaction using 4-hydroxybenzonitrile. This compound was able to undergo deoxyfluorination using the TFPN additive and anhydrous TMAF when ran at room temperature for overnight or heated at 85° C. for two hours, with a product yield of ˜70% shown in Scheme 4. Following the success of this phenol with a strong electron withdrawing group, we test both sodium phenoxide, as an alternative of phenol for easier handling, and 4-(trifluoromethyl)phenol to gauge under what conditions deoxyfluorination would occur and how much product forms. To our surprise, both the phenoxide and trifluoromethyl substituted phenol only formed the ether intermediate, excess fluoride did not drive the reaction to completion as expected. Similarly, para-hydroxy-benzoaldehyde only forms the difluoromethyl substituted phenyl ether with TFPN additive (Scheme 2). With additional TFPN and anhydrous TMAF, this ether is stable without any sign of further reaction under heating.

This result indicates that difluoromethyl substituted phenol does not go through deoxyfluorination under current conditions. Based on these results, we hypothesized that a resonance-active strong electron withdrawing group (RA-EWG) is key for activating phenols towards deoxyfluorination using the TFPN additive. To test this, we utilized a commercial intermediate to the drug molecule Febuxostat (Scheme 4), which possesses a hydroxyl, an aldehyde, and a thiazole group on the phenyl ring.

Under our reaction conditions, we were able to observe deoxfluorination of both the phenol and aldehyde with confirmation by NMR and GC-MS. The negative result of the sodium phenoxide deoxyfluorination is not surprising as the lack of a withdrawing moiety to activate the ether ipso carbon towards S_{N}Ar follows previous observations. However, phenol with a para trifluoromethyl substituent, a relatively strong electron withdrawing group with a σₚ value of +0.54, was unable to be converted to the corresponding deoxyfluorination product. A possible working hypothesis is that the resonance activation of the strong electron-withdrawing group plays a key role in phenol deoxyfluorination. Under normal conditions, a CF₃ group can undergo negative hyperconjugation as a resonance form. Under our fluoride-rich environment, the formation of the negative hyperconjugation structure may not be favored or stable enough to exist for prolonged periods of time to get the deoxyfluorination reaction to occur. Furthermore, the para CF₃ group can only stabilize the Meisenheimer complex by its inductive effect after fluoride attacks the ipso carbon of the ether intermediate. In contrast, para cyano group, with a σₚ value of +0.66, provides much-needed resonance stability, on top of the inductive effect, for the Meisenheimer complex after the fluoride attacks the ipso carbon, leading to the final deoxyfluorination product in good yield.

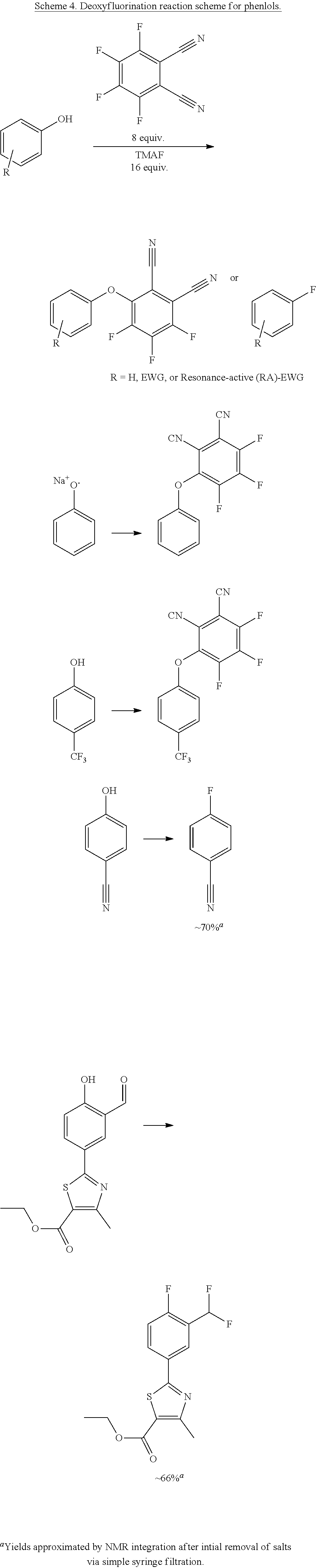

In Scheme 4, different phenols with substituents and final products are shown under the same equivalents and reaction conditions as for aldehydes and alcohols.

In summary, we reported a new deoxyfluorination methodology that is safe and selective for preparing monofluoromethyl and difluoromethyl derivatives from alcohols and aldehydes. Fluorinated electron-deficient aromatics such as TFPN can be effective reagents for transforming poor leaving groups into excellent ones and promoting fluorination. Work is currently underway in our group to develop new electron-deficient fluoroaromatics to expand the scope of this work, particularly to electron rich phenols and secondary alcohols, while providing a selection of reagents with even broader functional group tolerance.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Preparation of Acyl Fluorides from Carboxylic Acids

General Information.

Unless otherwise mentioned, all the chemicals were purchased from commercial sources and used without further purification. Spray-dried KF purchased from Sigma-Aldrich was used without further purification. In some cases, spray-dried KF was heated under vacuum at 200° C. for a week under dynamic vacuum. However, the pre-treatment of KF didn't show any difference on the experimental result. Anhydrous acetonitrile was dried over flame-dried 4 Å molecular sieves (4 Å). ¹H, ¹³C, and ¹⁹F NMR spectra were recorded on a Bruker 400 MHz NMR spectrometer with a SmartProbe™. Mass spectrum were recorded on a Shimadzu GC-2010 Plus mass spectrometer.

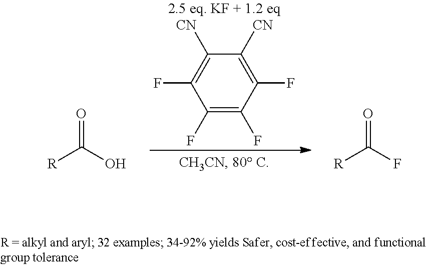

The procedure above provides a safer, cost-effective, and chemoselective deoxyfluorination reaction of carboxylic acids was achieved by using KF and highly electron-deficient fluoroarenes under mild conditions, giving acyl fluorides in moderate to excellent yield (34-92%) with a wide range of functional group compatibility. Directly utilizing KF for deoxyfluorination of carboxylic acids provides the most economical and safest pathway to access acyl fluorides, key intermediates for syntheses of amides, esters, peptides, and dry fluoride salts.

General procedure (A) for the preparation of acyl fluorides from carboxylic acids and NMR yield determination: The following procedure was used in the preparation of acyl fluorides from the corresponding carboxylic acids and sodium carboxylates. Inside an argon glovebox (H₂O<0.1 ppm; O₂<0.1 ppm), 1 mmol of carboxylic acid was weighed out into a screw cap vial. Next, 145 mg (2.5 mmol, 2.5 eq.) of KF and 240 mg (1.2 mmol, 1.2 eq.) of tetrafluorophthalonitrile were weighed out into the same vial, 2.5 mL of anhydrous acetonitrile (anhydrous propylene carbonate was used for high temperature experiments) was then added to the vial along with a small magnetic stir bar. In the case of using carboxylate salts (1 mmol) as the substrates, 87.15 mg (1.5 mmol, 1.5 eq.) of KF and 240 mg (1.2 mmol, 1.2 eq.) of tetrafluorophthalonitrile were used in the corresponding reaction set up. The vial was sealed with a screw-cap and transferred outside the glovebox for heating. The sample was then heated for 20-24 hours at 80-90° C. using a pre-heated oil bath (180° C./8 mins for high temperature experiments with PC as the solvent). After the reaction completion, the sample was cooled down to room temperature and 50 μL of 0.333 M TBAPF₆/CH₃CN (6.5 mg, 0.017 mmol) solution was added as an internal standard. The mixture was sealed and stirred for 10 minutes at room temperature before an aliquot was taken and analyzed by ¹⁹F NMR. The reported yields are determined by comparing the relative integration of internal standard signal (PF₆⁻) with the acyl fluoride product. ¹H and ¹⁹F NMR spectra were obtained by monitoring reaction mixture in CD₃CN solvent and/or by directly sampling the reaction mixture with CDCl₃ solvent without further purification. Upon mixing the reaction mixture with less polar CDCl₃ solvent, most side products KHF₂ and corresponding potassium phenoxide derivatives precipitated out of solution and separated with a syringe filter. Only small amounts of unreacted extra tetrafluorophthalonitrile was seen in the ¹⁹F NMR without further purification.

### Acetyl Fluoride

The title compound was prepared in 57% yield as determined by ¹⁹F NMR following the general preparation method. ¹H NMR (400 MHz, CD₃CN) δ 2.26 (d, J=7.41 Hz, 3H). ¹⁹F NMR (376 MHz, CD₃CN) δ 49.0 (q, J=7.43 Hz, 1F). Note: the yield determined here is likely lower than the accurate reaction yield due to operational loss of highly volatile acetyl fluoride (bp is 21° C.).

### Butanoyl Fluoride

The title compound was prepared in 82% yield as determined by ¹⁹F NMR following the general preparation method. ¹H NMR (400 MHz, CD₃CN) δ 8.05 (dd, J=8.32, 1.14 Hz, 2H), 7.80 (tt, J=7.60, 1.3 Hz, 1H), 7.64-7.57 (m, 2H). ¹⁹F NMR (376 MHz, CD₃CN) δ 16.5 (s, 1F). MS (EI) calculated for [M]⁺.: C₇H₅FO=124, found=124.

### 4-Ethylbenzoyl Fluoride

The title compound was prepared in 85% yield as determined by ¹⁹F NMR following the general preparation method. ¹H NMR (400 MHz, CD₃CN) δ 7.96 (d, J=8.4 Hz, 2H), 7.44 (d, J=7.8 Hz, 2H), 2.76 (q, J=7.60 Hz, 2H), 1.26 (t, J=7.60 Hz, 3H). ¹⁹F NMR (376 MHz, CD₃CN) δ 16.0 (s, 1F).

### 4 Acetylbenzoyl Fluoride

The title compound was prepared in 68% yield as determined by ¹⁹F NMR following the general preparation method. ¹H NMR (400 MHz, CD₃CN) δ 8.14 (d, J=8.56 Hz, 2H), 8.09 (d, J=8.56 Hz, 2H), 2.64 (s, 3H). ¹⁹F NMR (376 MHz, CD₃CN) δ 18.53 (s, 1F). MS (EI) calculated for [M]⁺.: C₉H₇FO₂=166, found=166.

### 4-Nitrobenzoyl Fluoride

The title compound was prepared in 79% yield as determined by ¹⁹F NMR following the general preparation method. ¹H NMR (400 MHz, CDCl₃) δ 8.38 (d, J=8.93 Hz, 2H), 8.25 (d, J=8.82 Hz, 2H). ¹⁹F NMR (376 MHz, CDCl₃) δ 21.28 (s, 1F). MS (EI) calculated for [M]⁺.: C₇H₄FNO₃=169, found=169.

### 3-Fluorobenzoyl Fluoride

The title compound was prepared in 92% yield as determined by ¹⁹F NMR following the general preparation method. ¹H NMR (400 MHz, CDCl₃) δ 7.85 (d, J=7.84 Hz, 1H), 7.74-7.68 (m, 1H), 7.57-7.50 (m, 1H), 7.46-7.38 (m, 1H). ¹⁹F NMR (376 MHz, CDCl₃) δ 19.2 (d, J=4.19 Hz, 1F), −110.74 (m, 1F). MS (EI) calculated for [M]⁺.: C₇H₄F₂O=142, found=142.

### 1-Adamantanecarboxylic Acid

The title compound was prepared in 79% yield as determined by ¹⁹F NMR following the general preparation method, using 1-adamantanecarboxylic acid (1 mmol, 180 mg), KF (2.5 mmol, 145 mg), TFPN (1.05 mmol, 210 mg). ¹H NMR (400 MHz, CDCl₃) δ 2.12-2.04 (m, 3H), 2.03-1.93 (m, 6H), 1.86-1.70 (m, 6H). ¹⁹F NMR (376 MHz, CDCl₃) δ 23.8 (s, 1F). MS (EI) calculated for [M]⁺.: C₁₁H₁₅FO=182, found=182.

### 4-tert-Butylbenzoyl Fluoride

The title compound was prepared in 78% yield as determined by ¹⁹F NMR following the general preparation method, using 4-tert-butylbenzoic acid (1 mmol, 178 mg), KF (2.5 mmol, 145 mg), TFPN (1.05 mmol, 210 mg). ¹H NMR (400 MHz, CDCl₃) δ 7.95-7.88 (m, 2H), 7.53-7.47 (m, 2H), 1.30 (s, 9H). ¹⁹F NMR (376 MHz, CDCl₃) δ 17.39 (s, 1F). MS (EI) calculated for [M]⁺.: C₁₁H₁₃FO=180, found=180.

### 4-Phenylbutanoyl Fluoride

The title compound was prepared in 67% yield as determined by ¹⁹F NMR following the general preparation method. ¹H NMR (400 MHz, CD₃CN) δ 7.36-7.28 (m, 2H), 7.28-7.20 (m, 3H), 2.70 (t, J=7.58 Hz, 2H), 2.59 (t, J=7.35 Hz, 2H), 1.96 (p, J=7.56 Hz, 2H). ¹⁹F NMR (376 MHz, CD₃CN) δ 43.38 (s, 1F).

### 4-(Dimethylamino)benzoyl Fluoride

The title compound was prepared in 79% yield as determined by ¹⁹F NMR following the general preparation method. ¹H NMR (400 MHz, CD₃CN) δ 7.79 (d, J=8.88 Hz, 2H), 6.71 (d, J=8.87 Hz, 2H), 3.06 (s, 6H). ¹⁹F NMR (376 MHz, CD₃CN) δ 10.38 (s, 1F). MS (EI) calculated for [M]⁺.: C₉H₁₀FNO=167, found=167.

### 4-Aminobenzoyl Fluoride

The title compound was prepared in 62% yield as determined by ¹⁹F NMR following the general preparation method, using 4-aminobenzoate (1 mmol, 159 mg), KF (1.5 mmol, 87 mg), TFPN (2.05 mmol, 410 mg). 50 μL TBAPF₆ standard solution added, 10 drops of reaction mixture was diluted with 0.5 ml of CD₃CN before taking NMR. ¹H NMR (400 MHz, CD₃CN) δ 7.76 (d, J=8.77 Hz, 2H), 6.71 (d, J=8.79 Hz, 2H), 5.23 (s, 2H). ¹⁹F NMR (376 MHz, CD₃CN) δ 10.38 (s, 1F). MS (EI) calculated for [M]⁺.: C₇H₆FNO=139, found=139.

### 4-Methoxybenzoyl Fluoride

The title compound was prepared in 91% yield as determined by ¹⁹F NMR following the general preparation method. ¹H NMR (400 MHz, CDCl₃) δ 7.98 (d, J=8.86 Hz, 2H), 6.98 (d, J=9.02 Hz, 2H), 3.89 (s, 3H). ¹⁹F NMR (376 MHz, CDCl₃) δ 15.8 (s, 1F). MS (EI) calculated for [M]⁺.: C₈H₇FO₂=154, found=154.

### 4-Vinylbenzoyl Fluoride

The title compound was prepared in 76% yield as determined by ¹⁹F NMR following the general preparation method without any heating and was stirred at r.t for a week, using 4-vinylbenzoic acid (1 mmol, 148 mg), KF (2.5 mmol, 145 mg), TFPN (1.2 mmol, 240 mg). ¹H NMR (400 MHz, CDCl₃) δ 7.98 (d, J=8.38 Hz, 2H), 7.53 (d, J=8.00 Hz, 2H), 6.77 (dd, J=17.63, 10.97 Hz, 1H), 5.93 (d, J=17.61, 7.0 Hz, 1H), 5.47 (d, J=10.91 Hz, 1H). ¹⁹F NMR (376 MHz, CDCl₃) δ 17.68 (s, 1F). MS (EI) calculated for [M]⁺.: C₉H₇FO=150, found=150.

### 4-Chlorobenzoyl Fluoride

The title compound was prepared in 70% yield as determined by ¹⁹F NMR following the general preparation method. ¹H NMR (400 MHz, CD₃CN) δ 8.07-8.01 (m, 2H), 7.66-7.60 (m, 2H). ¹⁹F NMR (376 MHz, CD₃CN) δ 17.01 (s, 1F). MS (EI) calculated for [M]⁺.: C₇H₄ClFO=158, found=158 (for ³⁵Cl), 160 (for ³⁷Cl).

### 4-Bromobenzoyl Fluoride

The title compound was prepared in 90% yield as determined by ¹⁹F NMR following the general preparation method. ¹H NMR (400 MHz, CDCl₃) δ 7.92-7.87 (m, 2H), 7.71-7.65 (m, 2H). ¹⁹F NMR (376 MHz, CDCl₃) δ 18.32 (s, 1F). MS (EI) calculated for [M]⁺.: C₇H₄BrFO=203, found=204 (for ⁸¹Br), 202 (for ⁷⁹Br).

### 4-Iodobenzoyl Fluoride

The title compound was prepared in 72% yield as determined by ¹⁹F NMR following the general preparation method. ¹H NMR (400 MHz, CDCl₃) δ 7.93-7.88 (m, 2H), 7.75-7.70 (m, 2H). ¹⁹F NMR (376 MHz, CDCl₃) δ 18.14 (s, 1F). MS (EI) calculated for [M]⁺.: C₇H₄FIO=250, found=250.

### 2-Naphthoyl Fluoride

The title compound was prepared in 77% yield as determined by ¹⁹F NMR following the general preparation method. ¹H NMR (400 MHz, CDCl₃) δ 8.64 (s, 1H), 8.03-7.89 (m, 4H), 7.71-7.65 (m, 1H), 7.65-7.58 (m, 1H). ¹⁹F NMR (376 MHz, CDCl₃) δ 17.93 (s, 1F). MS (EI) calculated for [M]⁺.: C₁₁H₇FO=174, found=174.

### 3-Pyridinecarbonyl Fluoride (Nicotinoyl Fluoride)

The title compound was prepared in 77% yield as determined by ¹⁹F NMR following the general preparation method. ¹H NMR (400 MHz, CDCl₃) δ 9.23 (d, J=2.00 Hz, 1H), 8.91 (dd, J=4.95, 1.69 Hz, 1H), 8.34-8.29 (m, 1H), 7.51 (dd, J=8.08, 4.90 Hz, 1H). ¹⁹F NMR (376 MHz, CDCl₃) δ 20.60 (s, 1F).

### Isonicotinoyl Fluoride

The title compound was prepared in 68% yield as determined by ¹⁹F NMR following the general preparation method. ¹H NMR (400 MHz, CDCl₃) δ 8.92-8.88 (m, 2H), 7.88-7.83 (m, 2H). ¹⁹F NMR (376 MHz, CDCl₃) δ 20.93 (s, 1F).

### Methyl-4-(fluorocarbonyl)benzoate

The title compound was prepared in 78% yield as determined by ¹⁹F NMR following the general preparation method, using monomethyl terephthalate (1 mmol, 180 mg), KF (2.5 mmol, 145 mg), TFPN (1.05 mmol, 210 mg). ¹H NMR (400 MHz, CDCl₃) δ 8.14 (d, J=8.28 Hz, 2H), 8.08 (d, J=8.40 Hz, 2H), 3.92 (s, 3H). ¹⁹F NMR (376 MHz, CDCl₃) δ 19.86 (s, 1F). MS (EI) calculated for [M]⁺.: C₉H₇FO₃=182, found=182.

### 4-Formylbenzoyl Fluoride

The title compound was prepared in 75% yield as determined by ¹⁹F NMR following the general preparation method. After reaction was finished, 50 μL of 0.333 M TBAFP₆/CH₃CN standard solution was added into the vial. ¹H NMR (400 MHz, CD₃CN) δ 10.15 (s, 1H), 8.27-8.22 (m, 2H), 8.12-8.07 (m, 2H). ¹⁹F NMR (376 MHz, CD₃CN) δ 18.82 (s, 1F). MS (EI) calculated for [M]⁺.: C₈H₅FO₂=152, found=152.

### 4-(Trifluoromethyl)benzoyl Fluoride

The title compound was prepared in 81% yield as determined by ¹⁹F NMR following the general preparation method. After reaction was finished, 50 μL of 0.333 M TBAFP₆/CH₃CN standard solution was added into the vial. ¹H NMR (400 MHz, CD₃CN) δ 8.24 (d, J=8.32 Hz, 2H), 7.93 (d, J=8.29 Hz, 2H). ¹⁹F NMR (376 MHz, CD₃CN) δ 18.52 (s, 1F), 64.05 (s, 3F). MS (EI) calculated for [M]⁺.: C₈H₄F₄O=192, found=192.

### 4-Ethynylbenzoyl Fluoride

The title compound was prepared in 72% yield as determined by ¹⁹F NMR following the general preparation method. ¹H NMR (400 MHz, CD₃CN) δ 8.05 (d, J=8.36 Hz, 2H), 7.70 (d, J=8.41 Hz, 2H), 3.75 (s, 1H). ¹⁹F NMR (376 MHz, CD₃CN) δ 17.19 (s, 1F). MS (EI) calculated for [M]⁺.: C₉H₅FO=148, found=148.

### 2, 6-Dichlorobenzoyl Fluoride

The title compound was prepared in 60% yield as determined by ¹⁹F NMR following the general preparation method. ¹H NMR (400 MHz, CD₃CN) δ 7.60-7.48 (m, 3H). ¹⁹F NMR (376 MHz, CD₃CN) δ 52.06 (s, 1F). MS (EI) calculated for [M]⁺.: C₇H₃Cl₂FO=192 (for ³⁵Cl), found=192 (for ³⁵Cl), 194 (for ³⁵Cl, ³⁷Cl), 196 (for ³⁷Cl).

### 2-Methoxybenzoyl Fluoride

The title compound was prepared in 73% yield as determined by ¹⁹F NMR following the general preparation method. ¹H NMR (400 MHz, CD₃CN) δ 7.90 (dd, J=7.90, 1.50 Hz, 1H), 7.75-7.68 (m, 1H), 7.19 (d, J=8.55 Hz, 1H), 7.09 (t, J=7.63 Hz, 1H), 3.94 (s, 3H). ¹⁹F NMR (376 MHz, CD₃CN) δ 30.60 (s, 1F). MS (EI) calculated for [M]⁺.: C₈H₇FO₂=154, found=154.

### 4-(Dipropylsulfamoyl)benzoyl Fluoride (Probenecid Fluoride)

The title compound was prepared on the bench-top with 34% yield following the general preparation method with Probenecid (4 mmol, 1.14 g), KF (10 mmol, 0.58 g), TFPN (4.2 mmol, 0.84 g) and 8 mL of CH₃CN. The reaction mixture was first concentrated to 2 ml of total volume and then quenched with a mixture of 40 ml of Hexane and 10 ml of DCM. Then the resulting mixture was passed through a short a short silica plug and washed silica plug with an 1:1 hexanes/DCM mixture. The filtrate was then concentrated under reduced pressure to reach saturation at about room temperature. Then the concentrated filtrate was cooled down to −18° C. for 15 mins followed by suction filtration to collect the final solid product. Yield, 390 mg. ¹H NMR (400 MHz, CDCl₃) δ 8.22-8.17 (m, 2H), 8.0-7.95 (m, 2H), 3.14 (t, J=7.65 Hz, 4H), 1.58 (h, J=7.74 Hz, 4H), 0.89 (t, J=7.4 Hz, 6H). ¹⁹F NMR (376 MHz, CDCl₃) δ 20.2 (s, 1F). ¹³C NMR (100 MHz, CDCl₃) δ 156.0 (d, J 346.1 Hz), 146.7, 132.1 (d, J 3.7 Hz), 128.1 (d, J 62.1 Hz), 127.5, 50.0, 22.0, 11.1. MS (EI) calculated for [M]⁺.: C₁₃H₁₈NO₃FS=287, found=287.

### (1-Benzoyl-5-methoxy-2-methyl-1H-indol-3-yl)acetyl Fluoride (Indomethacin Fluoride)

The title compound was prepared on the bench-top with 47% yield following the general preparation method with Indomethacin (1 mmol, 358 mg), KF (2.5 mmol, 145 mg), TFPN (1.05 mmol, 210 mg). Upon the completion of the reaction, the reaction mixture was quenched with 10 ml of 1:1 hexane and DCM mixture. Then the resulting mixture was filtrated over a short pad of silica using an 1:1 hexanes/DCM mixture as the eluent. The filtrate was then concentrated under reduced pressure to saturation about room temperature. The collected filtrate was cooled down to −18° C. for 15 mins followed by suction filtration to collect the final solid product. Yield, 170 mg. ¹H NMR (400 MHz, CDCl₃) δ 7.73-7.67 (m, 2H), 7.54-7.48 (m, 2H), 6.91 (d, J=2.35 Hz, 1H), 6.87 (d, J=9.0 Hz, 1H), 6.72 (dd, J=9.0, 2.4, 1H), 3.89 (d, J=2.45 Hz, 2H), 3.87 (s, 3H), 2.44 (s, 3H). ¹⁹F NMR (376 MHz, CDCl₃) δ 44.41 (s, 1F). ¹³C NMR (100 MHz, CDCl₃) δ 168.2, 160.5 (d, J=364.2 Hz), 156.2, 139.6, 137.0, 133.5, 131.3, 130.7, 130.0, 129.2, 115.1, 112.1, 109.2 (d, J=2.2 Hz), 100.7, 55.7, 28.3 (d, J=58.0 Hz), 13.2.

### (2S)-2-(6-methoxynaphthalen-2-yl)propanoyl Fluoride (Naproxen Fluoride)

The title compound was prepared on the bench-top with 42% yield (40% with PC high temperature experiment) following the general preparation method with Naproxen (1 mmol, 230 mg), KF (2.5 mmol, 145 mg), TFPN (1.05 mmol, 210 mg). The reaction was quenched with 10 ml of 1:1 hexane and DCM mixture. The resulting mixture was then passed through a short pad of silica using hexane as the eluent. The collected filtrate was then concentrated under reduced pressure to saturation at about room temperature. Then the concentrated filtrate was cooled down to −18° C. for 15 mins followed by suction filtration to collect the final solid product. Yield, 98 mg for reaction done in CH₃CN, and 93 mg for reaction done in PC. Due to higher polarity of the PC than acetonitrile, we used a mixture of 42 ml of hexane and 7 ml of DCM to quench the reaction while other steps were remaining the same as for the reaction done in acetonitrile. ¹H NMR (400 MHz, CDCl₃) δ 7.76 (d, J=21.1 Hz, 1H), 7.76 (d, J=3.4 Hz, 1H), 7.70 (d, J=1.7 Hz, 1H), 7.39 (dd, J=8.5, 1.9 Hz, 1H), 7.20 (dd, J=8.9, 2.5 Hz, 1H), 7.16 (d, J=2.5 Hz, 1H), 4.03 (q, J=7.1 Hz, 1H), 3.95 (s, 3H), 1.69 (dd, J=7.2, 0.8 Hz, 3H). ¹⁹F NMR (376 MHz, CDCl₃) δ 39.63 (s, 1F). ¹³C NMR (100 MHz, CDCl₃) δ 168.2, 160.5 (d, J=364.2 Hz), 156.2, 139.6, 137.0, 133.5, 131.3, 130.7, 130.0, 129.2, 115.1, 112.1, 109.2 (d, J=2.2 Hz), 100.7, 55.7, 28.3 (d, J=58.0 Hz), 13.2. MS (EI) calculated for [M]⁺.: C₁₄H₁₃FO₂=232, found=232.

### 2-(3-cyano-4-isobutoxyphenyl)-4-methylthiazole-5-carbonyl Fluoride (Febuxostat Fluoride)

The title compound was prepared on the bench-top with 76% (40% with PC high temperature experiment) yield following the general preparation method with Febuxostat (1 mmol, 316 mg), KF (2.5 mmol, 145 mg), TFPN (1.05 mmol, 210 mg), and 8 ml of CH₃CN. The reaction mixture was first condensed to 2 ml of total volume and then quenched with a mixture of 13.5 ml of Hexane and 2.5 ml of DCM. The resulting mixture was passed through a short pad of silica using an 1:1 hexanes/DCM mixture as the eluent. The filtrate was then concentrated under reduced pressure to saturation point at room temperature. The concentrated filtrate was then cooled down to −18° C. for 15 mins followed by suction filtration to collect the final solid product. Yield, 241 mg for reaction done in CH₃CN, and 124 mg for reaction don in PC. Due to the higher polarity of the PC than acetonitrile, we use a mixture of 42 ml of hexane and 7 ml of DCM to quench the reaction while other steps remained the same as for the reaction done in acetonitrile. ¹H NMR (400 MHz, CDCl₃) δ 8.25 (d, J=2.3 Hz, 1H), 8.13 (dd, J=8.9, 2.4 Hz, 1H), 7.06 (d, J=8.9 Hz, 1H) 3.94 (d, J=6.5 Hz, 2H), 2.82 (s, 3H), 2.24 (non, J=6.6 Hz, 1H), 1.12 (d, J=6.7 Hz, 6H). ¹⁹F NMR (376 MHz, CDCl₃) δ 37.63 (s, 1F). ¹³C NMR (100 MHz, CDCl₃) δ 170.7, 166.6 (d, J=6.6 Hz), 163.1, 152.0 (d, J=329.5 Hz), 132.9, 132.5, 125.2, 115.5, 115.1, 112.8, 103.3, 75.8, 28.2, 19.0, 17.8 (d, J=2.72 Hz). MS (EI) calculated for [M]⁺.: C₁₆H₁₅FN₂O₂S=318, found=318.

### 2-(3-benzoylphenyl)propionyl Fluoride (Ketoprofen Fluoride)

The title compound was prepared in 65% yield as determined by ¹⁹F NMR following the general preparation method, using Ketoprofen (1 mmol, 254 mg), KF (2.5 mmol, 145 mg), TFPN (1.05 mmol, 210 mg). ¹H NMR (400 MHz, CD₃CN) δ 7.81-7.74 (m, 3H), 7.72 (dt, J=7.64, 1.40 Hz, 1H), 7.70-7.60 (m, 2H), 7.58-7.50 (m, 3H), 4.17 (q, J=7.21 Hz, 1H), 1.60 (d, J=7.23, 3H). ¹⁹F NMR (376 MHz, CD₃CN) δ 39.46 (s, 1F). MS (EI) calculated for [M]⁺.: C₁₆H₁₃FO₂=256, found=256.

### 4-(2,3-dicyano-4,5,6-trifluorophenoxy)benzoyl Fluoride

The title compound was prepared in 72% yield in the form of TFPN-substituted product as determined by ¹⁹F NMR following the general preparation method, using sodium 4-hydroxybenzoate (1 mmol, 160 mg), KF (3.5 mmol, 203 mg), TFPN (2.2 mmol, 440 mg). The final product showed mixed results in NMR as GC indicated the product as a reaction intermediate. The main product is identified by GC with 72% yield. ¹H NMR (400 MHz, CDCl₃) δ 8.10 (d, J=8.80 Hz, 2H), 7.13 (d, J=8.78 Hz, 2H). ¹⁹F NMR (376 MHz, CDCl₃) δ 18.18 (s, 1F), −117.29 (dd, J=11.27, 8.25 Hz, 1F), −125.80 (dd, J=20.54, 10.94 Hz, 1F), −132.66 (dd, J=20.45, 8.77 Hz, 1F). MS (EI) calculated for [M]⁺.: C₁₅H₄F₄N₂O₂=320, found=320. The constitutional isomer of the product was also found in GC-MS with the same fragmentations.

General procedure (B). Example reaction for making compounds 1 and 2 shown below: 2 eq. of sodium 1,1-dicyanoethylene-2,2-dithiolate and 1 eq. of pentaflurobenzonitrile was mixed together in acetonitrile in a glovebox filled with nitrogen, then the reaction vial was sealed and heated at 80° C. and monitored by ¹⁹F NMR. The product 1 and 2 were identified by ¹⁹F NMR where compound 1 showed a ¹⁹F NMR signal at −100 ppm and compound 2 showed a ¹⁹F NMR signal at −105 ppm in acetonitrile. This mixed products 1 and 2 can be used together with the fluoride salts in deoxyfluorination reaction without separation.

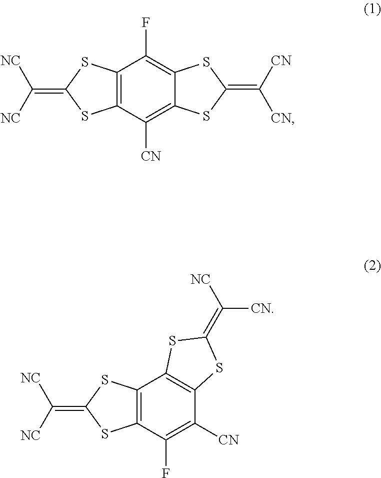 [SMILES: N#CC(C#N)=C1Sc2c(F)c3c(c(C#N)c2S1)SC(=C(C#N)C#N)S3 ; N#CC(C#N)=C1Sc2c(F)c(C#N)c3c(c2S1)SC(=C(C#N)C#N)S3]

Similarly, compounds 3 and 4 were prepared from pentafluoropyridine and sodium 1,1-dicyanoethylene-2,2-dithiolate with the same procedure stated above. They can be used together with fluoride salts for the corresponding deoxyfluorination reactions.

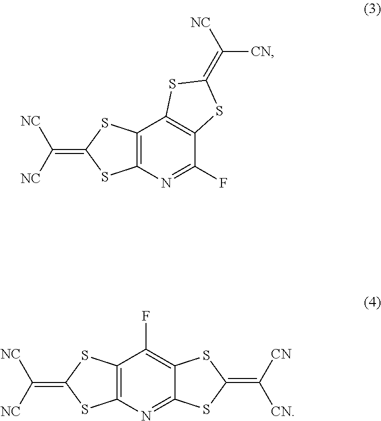 [SMILES: N#CC(C#N)=C1Sc2nc(F)c3c(c2S1)SC(=C(C#N)C#N)S3 ; N#CC(C#N)=C1Sc2nc3c(c(F)c2S1)SC(=C(C#N)C#N)S3]

### Example 2. Preparation Monofluoromethyl and Difluoromethyl Compounds from Alcohols and Aldehydes

The procedure described below provides an alternative safe and selective deoxyfluorination reagent and method for the preparation of di- and monofluorinated methyl moieties as well as aromatic fluorine from aldehydes, alcohols, and phenols, respectively, by utilizing an electron-deficient fluoroaromatic, tetrafluorophthalonitrile (TFPN), and anhydrous tetramethylammonium fluoride (TMAF). The efficacy of this method is in stark contrast to conventional deoxyfluorination reagents, such as diethylaminosulfur trifluoride (DAST), which places strict restrictions on reaction conditions and functional group tolerance. Unlike deoxyfluorination with DAST, this reaction can be done at both room and elevated temperatures without risks of explosive decomposition and leaves most functional groups unchanged upon completion. In addition, the byproducts produced can be safely disposed under standard laboratory conditions.

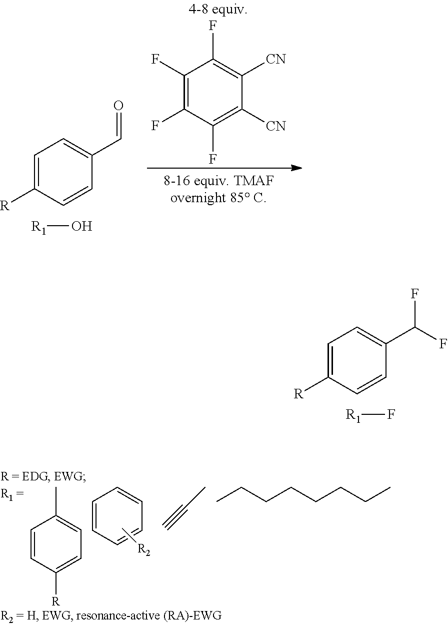

General Procedure:

Inside a nitrogen glovebox, 0.0500 g (0.00025 mol, 4 eq.) of tetrafluorophthalonitrile was weighed out into a vial. Next, 0.0466 g (0.0005 mol, 8 eq.) dried tetramethylammonium fluoride was weighed out and added into a vial. The solid samples were carefully transferred into a NMR tube followed by 0.25 mL of non-deuterated acetonitrile. Upon addition of the solvent, the solid samples began to dissolve and turn into a bright yellow coloration. Then, 1 eq. of substrate added into the NMR tube along with the remaining 0.25 mL of solvent. Typically, upon addition of the substrate the bright yellow solution would undergo a variety of color changes such as, but not limited to, dark brown, green, blue, red, and even purple for some substrates. This solution colors would darken after several minutes of sitting at room temperature. Finally, the NMR tube would be capped with a rubber septum and wrapped evenly with parafilm to be transferred out of the glovebox. These samples would be placed inside of an 85° C. oil bath for heating overnight. Upon heating these samples would almost all turn a dark red coloration within a minute or two of heating. Once the reactions were finished, the tubes would be cleaned of oil with ethyl acetate and a no-D NMR would be ran to assess the degree of deoxyfluorination. Note: should an acidic proton, such as a hydroxyl moiety, be present, 2 eq. of spray-dried potassium fluoride was added to the substrate before transferring into the main NMR tube.

Workup of the samples involved quenching the reaction into a less polar solvent, such as dichloromethane, and transferring the solvent to a small plastic syringe filled with silica and fitted with a syringe filter at the bottom. The silica would be washed with small quantities of less polar solvent to remove any remaining product while leaving majority of the salt byproducts on the column, including the phenoxide product and bifluoride. Due to the highly volatile nature of the deoxyfluorinated products, rotary evaporation was not performed for majority of reported substrates with the exception of the naphthalene, anthracene, and pyrene substrates. Therefore, confirmation of the worked up deoxyfluorination reactions was concluded with the use of a Shimadzu GC-MS set with an initial starting temperature of 30° C., injection temperature of 250° C., heating rate of 20° C./min until reaching and maintaining the temperature at 280° C. for approximately 35-40 minutes. This was used to not only verify the success of the deoxyfluorination, but also determine the yields based on integration.

### Deoxyfluorination Product Characterization

### 4-(Difluoromethyl)benzonitrile

¹H-NMR (400 MHz, CD₃CN): δ=7.87 ppm (d, J=7.53 Hz, 2H), 7.72 ppm (d, J=7.53 Hz, 2H), 6.89 ppm (t, J=56.1 Hz, 1H). ¹⁹F-NMR (376 MHz, CD₃CN): δ=−113.7 ppm (d, J=56.45 Hz, 2F). GCMS (EI, m/z): [M.⁺] calcd. for C8H5F2N, 153; found 152. Yield: 99% (Based on GC-MS integration)

### 4-(Difluoromethyl)-N,N-Dimethylaniline

¹H-NMR (400 MHz, CD₃CN): δ=7.36 ppm (d, J=8.02 Hz, 2H), 6.76 ppm (d, J=8.00 Hz, 2H), 6.67 ppm (t, J=57.13 Hz, 1H), 2.96 ppm (s, 6H). ¹⁹F-NMR (376 MHz, CD₃CN): δ=−106.6 ppm (d, J=57.00 Hz, 2F). GCMS (EI, m/z): [M.⁺] calcd. for C9H11F2N, 171; found 170. Yield: 85% (Based on GC-MS integration)

### 4-(Difluoromethyl)phenol

¹H-NMR (400 MHz, CDCl3): δ=7.55 ppm (d, J=9.01 Hz, 2H), 7.09 ppm (d, J=9.01 Hz, 2H), 6.66 ppm (t, J=56.20 Hz, 1H). ¹⁹F-NMR (376 MHz, CDCl3): δ=−110.34 ppm (d, J=56.27 Hz, 2F). GCMS (EI, m/z): [M.⁺] calcd. for C15H5F5N2O, 324; found 324. Yield: 100% (Based on GC-MS; existing as additive substituted derivative)

### Methyl 4-(difluoromethyl)benzoate

¹H-NMR (400 MHz, CD3CN): δ=8.07 ppm (d, J=7.73 Hz, 2H), 7.65 ppm (d, J=7.70 Hz, 2H), 6.87 ppm (t, J=56.30 Hz, 1H), 3.90 ppm (s, 3H). ¹⁹F-NMR (376 MHz, CD3CN): δ=−112.76 ppm (d, J=56.21 Hz, 2F). GCMS (EI, m/z): [M.⁺] calcd. for C9H8F2O2, 186; found 186. Yield: 90% (Based on GC-MS integration).

### 4-(Fluoromethyl)benzonitrile

¹H-NMR (400 MHz, CDCl3): δ=7.61 ppm (d, J=7.89 Hz, 2H), 7.39 ppm (d, J=7.89 Hz, 2H), 5.36 ppm (d, J=46.99 Hz, 2H). ¹⁹F-NMR (376 MHz, CDCl3): δ=−214.94 ppm (t, J=46.99 Hz, 1F). GCMS (EI, m/z): [M.⁺] calcd. for C8H6FN, 135; found 134. Yield: 100% (Based on GC-MS).

### 4-(Fluoromethyl)toluene

¹H-NMR (400 MHz, CDCl3): δ=7.18 ppm (m), 5.29 ppm (d, J=47.95 Hz, 2H), 1.85 ppm (s, 3H). ¹⁹F-NMR (376 MHz, CDCl3): δ=−203.29 ppm (t, J=47.83 Hz, 1F). GCMS (EI, m/z): [M.⁺] calcd. for C8H9F2, 124; found 124. Yield: 100% (Based on GC-MS).

### Methyl 4-(fluoromethyl)benzoate

¹H-NMR (400 MHz, CDCl3): δ=8.03 ppm (d, J=7.71 Hz, 2H), 7.41 ppm (d, J=7.78 Hz, 2H), 5.42 ppm (d, J=47.42 Hz, 2H), 3.89 ppm (s, 3H). ¹⁹F-NMR (376 MHz, CDCl3): δ=−212.66 ppm (t, J=47.45 Hz, 1F). GCMS (EI, m/z): [M.⁺] calcd. for C9H9FO2, 168; found 168. Yield: 100% (Based on GC-MS).

### α,α-Difluorotoluene

¹H-NMR (400 MHz, CDCl3): δ=7.49 ppm (br), 7.41 ppm (br), 7.60 ppm (br), 6.68 ppm (t, J=56.45 Hz, 1H). ¹⁹F-NMR (376 MHz, CDCl3): δ=−110.6 ppm (d, J=56.56 Hz, 2F). GCMS (EI, m/z): [M.⁺] calcd. for C7H6F2, 128; found 127. Yield: 74% (Based on GC-MS integration).

### α-fluorotoluene

¹H-NMR (400 MHz, CDCl3): δ=7.31 ppm (br), 5.30 ppm (d, J=47.76 Hz, 2H). ¹⁹F-NMR (376 MHz, CDCl3): δ=−206.22 ppm (t, J=47.78 Hz, 1F). GCMS (EI, m/z): [M.⁺] calcd. for C7H7F, 110; found 109. Yield: 100% (Based on GC-MS).

### 4-(fluoromethyl)-α,α,α-trifluorotoluene

¹H-NMR (400 MHz, CDCl3): δ=7.64 ppm (d, J=7.87 Hz, 2H), 7.48 ppm (d, J=7.87 Hz, 2H), 5.43 ppm (d, J=47.05 Hz, 2H). ¹⁹F-NMR (376 MHz, CDCl3): δ=−62.6 ppm (s, 3F), −212.78 ppm (t, J=47.08 Hz, 1F). GCMS (EI, m/z): [M.⁺] calcd. for C8H6F4, 178; found 178. Yield: 100% (Based on GC-MS).

### 4-Fluoro-α,α-difluorotoluene

¹H-NMR (400 MHz, CDCl3): δ=7.51 ppm (dd, J=8.01 Hz, 2H), 7.14 ppm (t, J=8.01 Hz, 2H), 6.64 ppm (t, J=56.07 Hz, 1H). ¹⁹F-NMR (376 MHz, CDCl3): δ=−109.62 ppm (d, J=56.04 Hz, 2F), −109.66 ppm (m, 1F). GCMS (EI, m/z): [M.⁺] calcd. for C7H5F3, 146; found 145. Yield: 75% (Based on GC-MS integration).

### 4-Fluoro-α-fluorotoluene

¹H-NMR (400 MHz, CDCl3): δ=7.33-7.21 ppm (m, 2H), 7.05-6.95 ppm (m, 2H), 5.27 ppm (d, J=48.02 Hz, 2H). ¹⁹F-NMR (376 MHz, CDCl3): δ=−112.7 ppm (m, 1F), −203.74 (t, J=48.18 Hz, 1F). GCMS (EI, m/z): [M.⁺] calcd. for C7H6F2, 128; found 128. Yield: 100% (Based on GC-MS).

### 4-Methyl-α,α-difluorotoluene

¹H-NMR (400 MHz, CDCl3): δ=7.68 ppm (d, J=7.79 Hz, 2H), 7.34 ppm (d, J=7.79 Hz, 2H), 6.60 ppm (t, J=56.77 Hz, 1H), 1.76 ppm (s, 3H). ¹⁹F-NMR (376 MHz, CDCl3): δ=−109.69 ppm (d, J=56.21 Hz, 2F). GCMS (EI, m/z): [M.⁺] calcd. for C8H8F2, 142; found 142. Yield: 87% (Based on GC-MS integration).

### 4-(difluoromethyl)anisole

¹H-NMR (400 MHz, CDCl3): δ=7.41 ppm (t, J=8.89 Hz, 2H), 6.91 ppm (m, 2H), 6.59 ppm (t, J=56.75 Hz, 1H), 3.8 ppm (s, 3H). ¹⁹F-NMR (376 MHz, CDCl3): δ=−108.22 ppm (d, J=57.05 Hz, 2F). GCMS (EI, m/z): [M.⁺] calcd. for C8H8F2O, 158; found 158. Yield: 45% (Based on GC-MS integration).

### 4-(Fluoromethyl)anisole

¹H-NMR (400 MHz, CDCl3): δ=7.32 ppm (dd, J=8.86 Hz, 2.22 Hz, 2H), 6.91 ppm (d, J=8.72 Hz, 2H), 5.29 ppm (d, J=48.38 Hz, 2H). ¹⁹F-NMR (376 MHz, CDCl3): δ=−198.99 ppm (t, J=48.43 Hz, 1F). GCMS (EI, m/z): [M.⁺] calcd. for C8H9FO, 140; found 139. Yield: 100% (Based on GC-MS).

### 1-Fluorooctane

¹H-NMR (400 MHz, CDCl3): δ=4.43 ppm (tt, J=6.42 Hz, 1.38 Hz, 2H), 4.13 ppm (t, 6.50 Hz, 2H), 1.80 ppm (m), 1.45 ppm (m), 1.29 ppm (br), 0.89 ppm (t, J=6.88 Hz, 3H). ¹⁹F-NMR (376 MHz, CDCl3): δ=−217.93 ppm (tt, J=47.80 Hz, 24.83 Hz, 1F). GCMS (EI, m/z): [M.⁺] calcd. for C8H17F, 132; found 132. Yield: 92% (Based on GC-MS integration).

### 3-Fluoro-1-propyne

b.p (Adapted Stein & Brown method): 4.62° C. ¹H-NMR (400 MHz, CD3CN): δ=5.57 ppm (br, J=42.10 Hz, 2H), 2.91 ppm (br, 1H). ¹⁹F-NMR (376 MHz, CD3CN): δ=−217.6 ppm (t, J=42.88 Hz, 1F). Yield: 100% (Based on lack of starting material in NMR spectrum).

### 2-(difluoromethyl)pyridine

¹H-NMR (400 MHz, CDCl3): δ=8.63 ppm (dd, J=6.20 Hz, 1.39 Hz, 1H), 7.75 ppm (td, J=7.75 Hz, 1.86 Hz, 1H), 7.64 ppm (d, J=7.76 Hz, 1H), 7.32 ppm (d, J=7.76 Hz, 1H), 6.64 ppm (t, J=55.14 Hz, 1H). ¹⁹F-NMR (376 MHz, CDCl3): δ=−115.86 ppm (d, J=55.27 Hz, 2F). GCMS (EI, m/z): [M.⁺] calcd. for C6H5F2N, 129; found 129. Yield: 100% (Based on GC-MS)

### [(1E)-3,3-difluoroprop-1-en-1-yl]benzene (Cinnamaldehyde deoxyfluorination)

¹H-NMR (400 MHz, CDCl3): δ=7.51 ppm (d, J=8.06 Hz, 2H), 7.45 ppm (d, J=8.06 Hz, 2H), 7.38-7.32 ppm (m), 7.24-7.12 ppm (m), 7.02-6.78 ppm (m). ¹⁹F-NMR (376 MHz, CDCl3): δ=−109.07 ppm (d, J=57.17 Hz, 2F). GCMS (EI, m/z): [M.⁺] calcd. for C9H8F2, 154; found 154. Yield: 87% (Based on GC-MS integration).

### 3-(Difluoromethyl)thiophene

¹H-NMR (400 MHz, CDCl3): δ=7.37 ppm (m), 7.24 ppm (m), 7.83 ppm (br), 7.05 ppm (dd, J=5.26 Hz, 1.44 Hz 1H), 6.69 ppm (t, J=56.52 Hz, 1H). ¹⁹F-NMR (400 MHz, CDCl3): δ=−108.25 ppm (d, J=56.52 Hz, 2F). GCMS (EI, m/z): [M.⁺] calcd. for C5H4F2S, 134; found 133. Yield: 86% (Based on GC-MS integration).

### Methyl Protected Febuxostat Precursor

The methyl protection of the Febuxostat precursor was accomplished by modification of the existing procedure with methyl iodide instead of isobutyl bromide. NMR and GC-MS confirmed the product and a yield of 99.5%. Deoxyfluorination followed the same general procedure used in the previous experiments. ¹H-NMR (400 MHz, CDCl3): δ=10.5 ppm (s, 1H), 8.38 ppm (d, J=2.41 Hz, 1H), 8.25 ppm (dd, J=8.7 Hz, 2.41 Hz, 1H), 7.11 ppm (d, J=8.77 Hz, 1H), 4.37 ppm (q, J=7.08 Hz, 2H), 4.04 ppm (s, 3H), 2.80 ppm (s, 3H), 1.41 ppm (t, J=7.12 Hz, 3H).

### Deoxyfluorination of Methyl Protected Febuxostat Precursor

¹H-NMR (400 MHz, CDCl3): δ=7.93 ppm (d, J=2.07 Hz 1H), 7.88 ppm (dd, J=8.68 Hz, 2.21 Hz, 1H), 7.02 ppm (d, J=8.55 Hz, 1H), 6.91 ppm (t, J=55.22 Hz, 1H), 6.82 ppm (s), 4.26 ppm (q, J=7.29 Hz), 3.92 ppm (s). ¹⁹F-NMR (376 MHz, CDCl3): δ=−116.63 ppm (d, J=55.14 Hz, 2F). GCMS (EI, m/z): [M.⁺] calcd. for C15H15F2NO3S, 327; found 327. Yield: 76% (Based on GC-MS integration)

### 4-Fluorobenzonitrile

¹H-NMR (400 MHz, CDCl3): δ=7.60-7.54 ppm (m, 2H), 7.09-6.98 ppm (m, 2H). ¹⁹F-NMR (376 MHz, CDCl3): δ=−103.54 ppm (tt, J=8.18 Hz, 1F). GCMS (EI, m/z): [M.⁺] calcd. for C7H4FN, 121; found 121 Yield: ˜70% (Based on NMR integration).

### Deoxyfluorination of Febuxostat Intermediate

¹H-NMR (400 MHz, CD3CN): δ=8.22 ppm (d, J=6.71 Hz, 1H), 8.17 ppm (m, 1H), 7.39 ppm (t, J=9.36 Hz), 7.06 ppm (t, J=54.03 Hz, 1H), 4.35 ppm (q, J=7.05 Hz, 2H), 2.73 (s, 3H), 1.28 ppm (t, J=7.09 Hz, 3H). ¹⁹F-NMR (376 MHz, CD3CN): −115.48 ppm (dd, J=54.06 Hz, 4.11 Hz, 2F), −121.25 ppm (m, 1F). GCMS (EI, m/z): [M.⁺] calcd. for C14H12F3NO2S, 315; found 315. Yield: ˜66% (Based on NMR integration).

### 2-(Difluoromethyl)naphthalene

¹H-NMR (400 MHz, CDCl3): δ=8.0-7.9 ppm (m), 7.65-7.58 ppm (m), 6.84 ppm (t, J=56.44 Hz, 1H). ¹⁹F-NMR (376 MHz, CDCl3): δ=−109.87 ppm (d, J=56.43 Hz, 2F). GCMS (EI, m/z): [M.⁺] calcd for C11H8F2, 178; found 178. Yield: 0.079 g (69%, colorless solid).

### 9-(Difluoromethyl)anthracene

¹H-NMR (400 MHz, CDCl3): δ=8.66 ppm (s, 1H), 8.49 ppm (d, J=8.62 Hz, 2H), 8.08 ppm (d, J=8.63 Hz, 2H), 8.02 ppm (t, J=53.90 Hz, 1H), 7.62 ppm (d, J=8.30 Hz, 2H), 7.55 ppm (d, J=8.26 Hz, 2H). ¹⁹F-NMR (376 MHz, CDCl3) δ=−106.60 ppm (d, J=53.95 Hz, 2F). GCMS (EI, m/z): [M.⁺] calcd for C15H10F2, 228; found 228. Yield: 0.063 g (˜57%, yellow solid after purification).

### 1-(Difluoromethyl)pyrene

¹H-NMR (400 MHz, CDCl3): δ=8.41 ppm (d, J=9.42 Hz, 1H), 8.30-8.21 ppm (m), 8.17 ppm (d, J=9.24, 1H), 8.13-8.06 ppm (m), 7.49 (t, J=55.45 Hz, 1H). ¹⁹F-NMR (376 MHz, CDCl3): −108.81 ppm (d, J=55.39 Hz, 2F). GCMS (EI, m/z): [M.⁺] calcd for C17H10F2, 252; found 252. Yield: 0.066 g (˜60%, orange solid after purification).

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method for deoxyfluorination comprising contacting an organic substrate, fluoride salt, electron-deficient fluoroaromatic, and polar aprotic organic solvent at a reaction temperature above −80° C.;
wherein the electron-deficient fluoroaromatic comprises one or more additional electron withdrawing group (EWG);
the substrate and fluoroaromatic form an intermediate;
the substrate comprises an oxygen moiety, the oxygen moiety forms an intermediate C—O bond via ipso substitution of a fluoro substituent on the aromatic moiety of the fluoroaromatic; and
the intermediate C—O bond breaks via nucleophilic attack by a fluoride ion at the substrate moiety of the intermediate to form a C—F bond;
wherein a fluorinated product is thereby formed by deoxyfluorination of the organic substrate.

2. The method of claim 1 wherein the fluoroaromatic is represented by Formula I or IB: 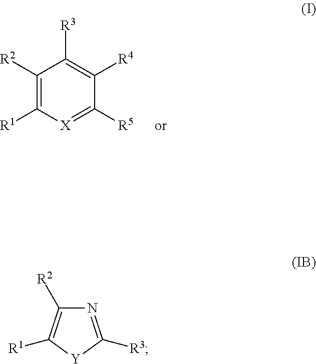
wherein
X is N or CR⁶ wherein R⁶ is F, CN, SF₅, or CₙF₂ₙ₊₁ wherein n is an integer equal to 1 or more;
Y is S or NR^{A} wherein R^{A} is H or —(C₁-C₆)alkyl;
R¹, R², R³, R⁴, and R⁵ are each independently F, CN, SF₅, or CₙF₂ₙ₊₁; or
R¹ to R⁶ are as defined above provided that R¹ and R² when taken together form a first heterocycle and R³ and R⁴ or R⁴ and R⁵ when taken together form a second heterocycle, wherein the first heterocycle and the second heterocycle are each independently an optional 5- or 6-membered heterocycle comprising the additional one or more EWG; and
provided at least one of R¹ to R⁶ is fluoro.

3. The method of claim 2 wherein the fluoroaromatic is a fluorophenylnitrile.

4. The method of claim 2 wherein the fluoroaromatic is a tetrafluorophthalonitrile.

5. The method of claim 2 wherein the fluoroaromatic is 3,4,5,6-tetrafluorophthalonitrile (TFPN):

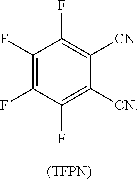

6. The method of claim 2 wherein the fluoroaromatic of Formula I is represented by Formula II: 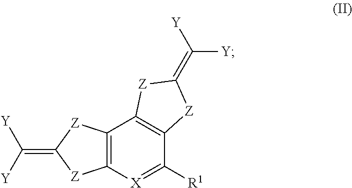
wherein
each Y is independently fluoro or cyano;
each Z is independently O or S; and
at least one of R¹ or R⁶ is F.

7. The method of claim 6 wherein the fluoroaromatic is 2,2′-(5-fluorobis([1,3]dithiolo)[4,5-b:4′,5′-d]pyridine-2,7-diylidene)dimalononitrile:

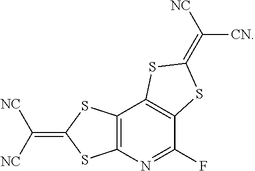 [SMILES: N#CC(C#N)=C1Sc2nc(F)c3c(c2S1)SC(=C(C#N)C#N)S3]

8. The method of claim 6 wherein the fluoroaromatic is 2,2′-(4-cyano-5-fluorobenzo[1,2-d:3,4-d′]bis([1,3]dithiole)-2,7-diylidene)dimalononitrile:

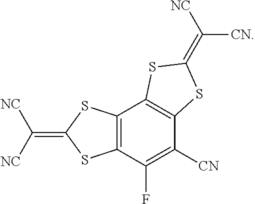 [SMILES: N#CC(C#N)=C1Sc2c(F)c(C#N)c3c(c2S1)SC(=C(C#N)C#N)S3]

9. The method of claim 2 wherein the fluoroaromatic of Formula I is represented by Formula III: 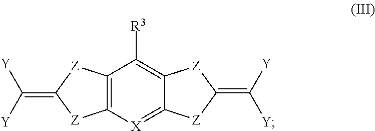
wherein
each Y is independently fluoro or cyano;
each Z is independently O or S; and
at least one of R³ or R⁶ is F.

10. The method of claim 9 wherein the fluoroaromatic is 2,2′-(8-fluorobis([1,3]dithiolo)[4,5-b:4′,5′-e]pyridine-2,6-diylidene)dimalononitrile:

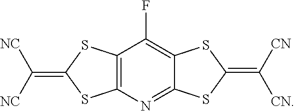 [SMILES: N#CC(C#N)=C1Sc2nc3c(c(F)c2S1)SC(=C(C#N)C#N)S3]

11. The method of claim 9 wherein the fluoroaromatic is 2,2′-(4-cyano-8-fluorobenzo[1,2-d:4,5-d′]bis([1,3]dithiole)-2,6-diylidene)dimalononitrile:

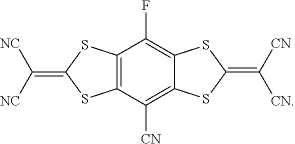 [SMILES: N#CC(C#N)=C1Sc2c(F)c3c(c(C#N)c2S1)SC(=C(C#N)C#N)S3]

12. The method of claim 1 wherein the organic substrate comprises an alcohol, phenol, aldehyde, carboxylic acid, or salt thereof.

13. The method of claim 1 wherein the fluoride salt is lithium fluoride, sodium fluoride, potassium fluoride, cesium fluoride, ammonium fluoride, tetramethylammonium fluoride, tetraethylammonium fluoride, tetrapropylammonium fluoride, tetrabutylammonium fluoride, or a fluoride isotope thereof.

14. The method of claim 1 wherein the organic solvent is acetonitrile, N-methyl-2-pyrrolidinone, propylene carbonate, dimethyl sulfoxide, tetrahydrofuran, or benzonitrile.

15. The method of claim 1 wherein the reaction temperature is about 50° C. to about 200° C.

16. The method of claim 1 wherein the fluorinated product comprises an alkyl fluoride, acyl fluoride, aryl fluoride, or heteroaryl fluoride.

17. The method of claim 1 wherein the fluorinated product comprises a difluoromethyl group.

18. The method of claim 1 wherein the fluoroaromatic is 3,4,5,6-tetrafluorophthalonitrile, the fluoride salt is potassium fluoride or tetramethylammonium fluoride, the organic solvent is acetonitrile, and the reaction temperature is about 75° C. to about 95° C.

19. A composition comprising a fluoroaromatic compound represented by Formula I: 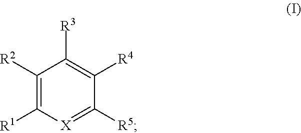
wherein
X is N or CR⁶ wherein R⁶ is F, CN, SF₅, or CₙF₂ₙ₊₁ wherein n is an integer equal to 1 or more;
R¹, R², R³, R⁴, and R⁵ are each independently F, CN, SF₅, or CₙF₂ₙ₊₁; or
R¹ to R⁶ are as defined above provided that R¹ and R² when taken together form a first heterocycle and R³ and R⁴ or R⁴ and R⁵ when taken together form a second heterocycle, wherein the first heterocycle and the second heterocycle are each independently an optional 5- or 6-membered heterocycle comprising an additional one or more electron withdrawing group (EWG); and
provided at least one of R¹ to R⁶ is fluoro; and
a fluoride salt.

20. A compound represented by Formula II or III: 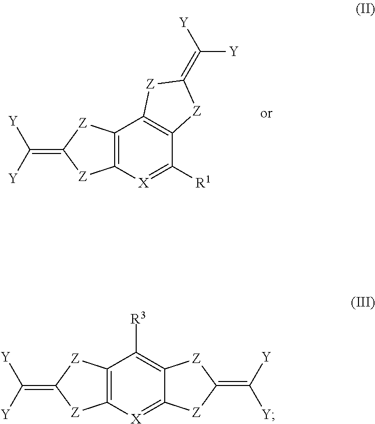
wherein
X is N or CR⁶;
R¹ or R³ is F, CN, SF₅, —OW, or CₙF₂ₙ₊₁ wherein n is an integer equal to 1 or more;
R⁶ is F, CN, SF₅, —OR⁷, or CₙF₂ₙ₊₁;
R⁷ is saturated or unsaturated alkyl, acyl, aryl, or heteroaryl;
each Y is independently fluoro or cyano;
each Z is independently O or S; and
provided at least one of R¹ or R⁶ is F or OR⁷, or one of R³ or R⁶ is F or OR⁷.

21. The compound of claim 20 wherein the compound is: [chemical structure]
